(19) United States
(12) Patent Application Publication
(10) Pub. No.: US 2019/0257757 A1
(43) Pub. Date: Aug. 22, 2019

(54) Breath Capture and Analysis System

(71) Applicant: Pulse Health LLC, Lake Oswego, OR (US)

(72) Inventors: Gerald Thomas, Springfield, OR (US); Juven Lara, Portland, OR (US); Brian Young, Portland, OR (US); Charles Noll, Portland, OR (US); Craig Carlsen, Vancouver, WA (US); James Ingle, Corvallis, OR (US)

(21) Appl. No.: 16/403,309

(22) Filed: May 3, 2019

Related U.S. Application Data
(63) Continuation of application No. 15/844,288, filed on Dec. 15, 2017.
(60) Provisional application No. 62/435,058, filed on Dec. 15, 2016.
(60) Provisional application No. 62/539,872, filed on Aug. 1, 2017.

(51) Int. Cl. G01N 21/64; A61B 5/097; G01N 30/74; G01N 30/36; G01N 30/34; A61B 5/08; A61B 5/00

(52) U.S. Cl. CPC G01N 21/6428; A61B 5/097; G01N 30/74; G01N 2030/027; G01N 30/34; A61B 5/082; A61B 5/0071; G01N 30/36

(57) ABSTRACT

Systems and methods directed to aldehyde detection are disclosed. An aldehyde detection system may capture aldehydes from a patient breath sample. Aldehydes of the breath sample may be used to form a mobile chromatography phase within an analysis device. The analysis device may include various modules configured to perform a high pressure liquid chromatography process that separates aldehydes according to size. A detection assembly may detect a relative value of the separated aldehydes. The analysis device may be configured to determine an aldehyde score or metric based on the detected aldehydes, which may assist in the diagnosis of certain medical conditions.

### CROSS-REFERENCE TO RELATED APPLICATIONS

This application is a continuation of U.S. patent application Ser. No. 15/844,288, filed Dec. 15, 2017, and titled “Aldehyde Analysis System and Method of Use,” which is a nonprovisional patent application of and claims the benefit of U.S. Provisional Patent Application No. 62/435,058, filed Dec. 15, 2016, and titled “Aldehyde Testing Device and Method of Use,” and U.S. Provisional Patent Application No. 62/539,872, filed Aug. 1, 2017, and titled “Methods and Systems for Aldehyde Detection,” the disclosures of which are hereby incorporated herein by reference in their entirety.

### FIELD

The described embodiments relate generally to diagnostic devices and methods of use. More particularly, the present embodiments relate to detecting chemical compounds, such as aldehydes, within a sample.

### BACKGROUND

Breath from a patient or user may contain aldehydes that can provide information on the general health and wellness of the patient. Aldehydes in breath (or urine, plasma, or headspace of biopsied cells) may be detected and analyzed to measure oxidation stress, among other characteristics, that may assist in a medical diagnosis of the patient. Many traditional detection systems and techniques suffer from significant drawbacks, including using multiple instruments, chemical containers, and other laboratory equipment that may lack integration and require attendance by a highly skilled or expert operator. Thus, there is a need for systems and techniques that can be used to integrate disparate components to automate and simplify aldehyde detection without limiting the functionality, accuracy, or reliability of the detection.

### SUMMARY

Embodiments of the present disclosure are directed to a breath analysis system for determining an aldehyde content of a breath sample.

In a first aspect, the present disclosure includes a breath analysis system. The breath analysis system includes a breath capture component having an internal volume. The breath analysis system further includes a cartridge attachable to the breath capture component and having a permeable layer. The breath analysis further includes an analysis device coupled with the cartridge opposite the breath capture component. The breath analysis device further includes a container received through an opening in the analysis device and having a group of internal chambers. The analysis device may be configured to draw a breath sample held within the internal volume of the breath capture component through the permeable layer. The analysis device may be further configured to determine an aldehyde content of the breath sample using a group of reagents contained by the internal chambers of the container.

In a second aspect, the present disclosure includes an analysis device for an aldehyde detection system. The analysis device includes an enclosure. The analysis device further includes a mixing volume positioned within the enclosure and configured to form a mobile chromatography phase using a breath sample and a group of reagents concealed by the enclosure. The analysis device further includes a column coupled with the mixing volume by a multi-position valve and having a stationary chromatography phase. The analysis device further includes a pump configured to push the mobile chromatography phase through the column using another reagent having a concentration controlled by a buffer. The analysis device further includes a detector configured to optically measure an aldehyde content output from the column. The analysis device further includes a display at least partially positioned within the enclosure and configured to depict a graphical output corresponding to the aldehyde content.

In a third aspect, the present disclosure includes a method for determining an aldehyde content of multiple breath samples. The method includes a first step of drawing a first breath sample of the multiple breath samples through a permeable membrane connected to an analysis device. The method further includes a second step of eluting the breath sample from the permeable membrane using a first reagent from a container positioned within the analysis device. The method further includes a third step of advancing the eluted breath sample through a column using a second reagent from the container. The method further includes a fourth step of detecting fluoresced particles at an output of the column corresponding to the aldehyde content of the breath sample. The method further includes a fifth step of repeating steps one through four for a second breath sample of the multiple breath samples. The container may include a quantity of the first reagent and the second reagent for at least each of the first breath sample and the second breath sample.

In a fourth aspect, the present disclosure includes an analysis device for an aldehyde detection system. The analysis device includes a sample capture module configured to retain aldehydes from a breath sample. The analysis device further includes a mixing module coupled to the sample capture module and configured to mix the retained aldehydes with a group of reagents. The analysis device further includes an injection module separated from the mixing module and configured to form a pressurized combination of another reagent and a buffer. The analysis device further includes a detection module configured to determine a value of the retained aldehydes. The detection module may determine the value of the retained aldehydes by receiving an output of the mixing module in a first configuration that loads a sample loop. The detection module may further determine the value of the retained aldehydes by, in response to loading a volume of the sample loop, receiving an output of the injection module in a second configuration that advances the loaded volume through a column. The detection module may further determine the value of the retained aldehydes by detecting a brightness of particles at an output of the column to determine the value of retained aldehydes.

In addition to the exemplary aspects and embodiments described above, further aspects and embodiments will become apparent by reference to the drawings and by study of the following description.

### BRIEF DESCRIPTION OF THE DRAWINGS

The disclosure will be readily understood by the following detailed description in conjunction with the accompanying drawings, wherein like reference numerals designate like elements.

FIG. 1 depicts an analysis device;

FIG. 2A depicts a breath capture component having a breath sample from a user;

FIG. 2B depicts the breath capture component of FIG. 2A attached to a cartridge;

FIG. 2C depicts a breath analysis system having the sample cartridge of FIG. 2B attached to an analysis device;

FIG. 2D depicts the breath analysis system in a configuration corresponding to an analysis of the breath sample;

FIG. 2E depicts the breath analysis system having a graphical output corresponding to a detection of chemical compounds within the breath sample;

FIG. 3 depicts a functional diagram of an analysis device;

FIG. 4 depicts an illustrative sample capture module of the analysis device;

FIG. 5 depicts an illustrative mixing module of the analysis device;

FIG. 6 depicts another embodiment of a mixing module of the analysis device;

FIG. 7 depicts an illustrative injection module of the analysis device;

FIG. 8 depicts an illustrative detection module of the analysis device;

FIG. 9A depicts a first configuration of a control value of the detection module;

FIG. 9B depicts a second configuration of a control value of the detection module;

FIG. 9C depicts a third configuration of a control value of the detection module;

FIG. 10A depicts a detection assembly of the detection module;

FIG. 10B depicts a brightness-time curve for detecting aldehydes;

FIG. 11 depicts a sample piping and instrument diagram for the analysis device;

FIG. 12A depicts a flow path of the sample piping and instrument diagram of FIG. 11;

FIG. 12B depicts another flow path of the sample piping and instrument diagram of FIG. 11;

FIG. 12C depicts another flow path of the sample piping and instrument diagram of FIG. 11;

FIG. 12D depicts another flow path of the sample piping and instrument diagram of FIG. 11;

FIG. 12E depicts another flow path of the sample piping and instrument diagram of FIG. 11;

FIG. 12F depicts another flow path of the sample piping and instrument diagram of FIG. 11;

FIG. 12G depicts another flow path of the sample piping and instrument diagram of FIG. 11;

FIG. 12H depicts another flow path of the sample piping and instrument diagram of FIG. 11;

FIG. 12I depicts another flow path of the sample piping and instrument diagram of FIG. 11;

FIG. 13 depicts a flow diagram for determining an aldehyde content of multiple breath samples;

FIG. 14 depicts an analysis device having an enclosure and a display;

FIG. 15 depicts an exploded view of the analysis device and a reagent container;

FIG. 16 depicts an illustrative connection between the analysis device and the reagent container of FIG. 15;

FIG. 17 depicts a cross-sectional view of the analysis device of FIG. 14, taken along line A-A of FIG. 14; and

FIG. 18 depicts a functional block diagram of a system including an analysis device.

The use of cross-hatching or shading in the accompanying figures is generally provided to clarify the boundaries between adjacent elements and also to facilitate legibility of the figures. Accordingly, neither the presence nor the absence of cross-hatching or shading conveys or indicates any preference or requirement for particular materials, material properties, element proportions, element dimensions, commonalities of similarly illustrated elements, or any other characteristic, attribute, or property for any element illustrated in the accompanying figures.

Additionally, it should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

The description that follows includes sample systems, methods, and apparatuses that embody various elements of the present disclosure. However, it should be understood that the described disclosure may be practiced in a variety of forms in addition to those described herein.

The present disclosure describes systems, devices, and techniques related to aldehyde detection and analysis. Aldehydes may include substantially any organic chemical compound characterized by a common (functional) group CHO (carbon, hydrogen, oxygen) structure bonded to an aldehyde group. Aldehydes may be detected and analyzed in breath (or other sample from a patient or user) to provide information on the general health and wellness of the patient. For example, a value (concentration or amount) of an aldehyde group having a particular characteristic, size, weight, or the like (such as an aldehyde designated by a C4, C5, C6, or the like) may be indicative of certain medical conditions or otherwise be used for medical diagnostics. However, a breath sample may be a heterogeneous mixture having aldehydes of various different designations. Detection may thus involve separating the sample by distinct aldehyde groups, often through sophisticated, multi-step chemical processes that may hinder the adaptability and repeatability of such techniques.

The breath analysis system of the present disclosure may mitigate such hindrances, thereby allowing for repeated detection of aldehydes by untrained technicians or clinical personnel. The amount and/or concentration of certain aldehydes within a breath sample may be used to diagnose certain health information about a patient or otherwise used in monitoring a patient's health, including incidences of cancer, tumors, or other malignant tissue within the patient's body. Further, although embodiments are described herein that analyze a breath sample taken from a patient, other embodiments may analyze different gases or fluids.

Broadly, an unattended high-pressure liquid chromatography (“HPLC”) process is integrated with an analysis device that converts a patient breath sample into a mobile (liquid) chromatography phase. The HPLC process may separate aldehydes of the mobile chromatography phase, as described herein, and be coupled to a detector that measures a value of the aldehydes according to molecule size. This may allow for an integrated approach that substantially automates aldehyde detection from breath capture to aldehyde score. In other embodiments, aldehydes may be measured, separated, or otherwise determined or distinguished from one another by any suitable chemical property, including sizes, shapes, hydrophobicity, hydrophilicty, charge, polarity, and so on. In this regard, the methods for aldehyde detection disclosed and described in U.S. Patent Application No. 62/539,872, filed Aug. 1, 2017, and titled “Methods and Systems for Aldehyde Detection,” are hereby incorporated by reference.

To facilitate the foregoing, the breath analysis system may include various components that cooperate to capture a patient breath sample, elute aldehydes from the breath sample, and determine an aldehyde content, among other functions. In one embodiment, the breath analysis system includes a breath capture component, such as an inflatable bag. A patient may exhale into the bag, causing the bag to inflate and retain a breath sample. The bag may be attachable to a cartridge having a permeable membrane (e.g., a silica or other like material) positioned along an interior flow path. An analysis device of the breath analysis system may be attached to the cartridge and used to pull or otherwise extract the breath sample through the permeable membrane (e.g., using a vacuum pump). The permeable membrane may retain aldehydes as the breath sample is drawn from the breath capture component. A container may be received by the analysis device and include one or more chemical compounds, reagents (e.g., methanol (“MeOH”), buffers, dyes, and/or other items that may facilitate manipulation of the retained aldehydes and detection by the analysis device.

Broadly, the analysis device may use a group of reagents from the container to determine an aldehyde content of the breath sample. For example, the analysis device may form an elution that captures the retained aldehydes of the permeable membrane. This may be used as an input to an HPLC process, described herein, that separates aldehydes according to molecule size. The analysis device may also include a display configured to depict a graphical output corresponding to a detected value of the separated aldehydes. In some cases, the output of the analysis device may be coupled with another electronic device, including over a wireless or distributed network, to facilitate diagnoses of a medical condition based on the detected aldehydes.

The analysis device may include various different modules that cooperate to determine the aldehyde content of a breath sample. Each module may be configured to execute one or more functions of a process (or separate processes) that converts the patient breath sample into a mobile (e.g., liquid) chromatography phase and detect aldehydes of the breath sample using an unattended HPLC process. In an embodiment, a sample capture module may elute retained aldehydes of the permeable membrane using one or more reagents from a reagent module, for example, such as by flushing the permeable membrane with MeOH 40% and/or other appropriate reagent or concentration. A mixing module, coupled with the sample capture module, may mix the eluted aldehydes with the same or different reagents of the reagent module to form the mobile chromatography phase. This may involve adding a catalyst, dye, calibrants, and so on to the elution and mixing with air agitation. An injection module, separated and parallel to the mixing module, may form a pressurized combination of a further reagent (e.g., including MeOH 10%-MeOH 100%) from the reagent module and a buffer used to control the concentration of reagent. The mobile chromatography phase output by the mixing module may be directed into the flow path of the pressurized output of the injection module, thereby allowing the injection module to push or pump the mobile chromatography phase through a static or stationary chromatography phase for aldehyde separation.

The analysis device may thus also include a detection module coupled with the mixing module and the injection module. The detection module may include a stationary chromatography phase. The stationary chromatography phase may be defined by a column having a high density silica structure. The detection module may be configured to load the mobile chromatography phase in a sample loop coupled with an inlet of the column (e.g., using a control valve, including the multi-position, multi-port valve, described herein) and allow the pressurized output of the injection module to advance the mobile chromatography phase through the silica structure of the column. The pressurized output of the injection module may have a concentration of reagent tuned to allow a particular size or designation of aldehyde through the column. For example, an initial concentration of the reagent may allow the smallest of the aldehyde groups to pass through the column; the concentration may be gradually increased (according to a predefined gradient ramp) to selectively allow increasing sizes of aldehyde groups to pass through. This effectively groups and separates aldehydes by size, so that the column outputs a slug or cluster of aldehydes all having a similar size or characteristic. In some embodiments, each aldehyde group will travel through the column separately without any overlap; in other embodiments, some overlap between aldehyde groups may occur and posts-processing of brightness data (or other data related to aldehyde detection) may be used to separate aldehyde group members from one another.

The detection module of the analysis device may thus be configured to measure a value (quantity, amount, or the like) of the aldehyde cluster output from the column. As described in greater detail below, the value of aldehydes may be detected using a laser or other excitation source that fluoresces a dye attached to the aldehyde groups as each are emitted from the column. A detector, for example, may measure an increase in brightness of the output of the column to facilitate a determination of an aldehyde content of the breath sample. A processing unit of the analysis device (or of another electronic device) may correlate the detected increase in brightness with a particular aldehyde group size or designation using the gradient ramp produced by the injection module. For example, aldehyde groups having a particular size or designation may progress through the column at a rate according to the gradient ramp controlled by the injection module (e.g., C4 aldehyde may require x seconds to progress through the column, whereas C5 aldehyde may require x+y seconds, and so forth based on the gradient ramp). Accordingly, the detection of fluoresced particles at the output of the column may be associated with the anticipated progression of certain aldehyde groups through the column, and thus used to determine a relative value of each aldehyde group in the breath sample. Put another way, the timing of aldehyde groups passing through the column may be used to determine what particular aldehydes are being detected, while the brightness of each such group may be used to determine an amount or concentration of aldehydes within each group. Thus, embodiments may determine relative and/or absolute concentrations of aldehydes within a user's breath sample.

It will be appreciated that the reagent module, breath capture module, mixing module, injection module, detection module, and so on may collectively represent a network of tubes, pumps, valves, sensors, and/or other mechanical components, instrumentation, and devices and so on that are used to perform the various functions of the modules described herein. As described herein, the modules may be self-contained and interconnected systems within a portable device. As such, rather than discrete systems, the modules may be coupled to one another (e.g., within the analysis device) and use common components of the system (e.g., a given pump or valve may be used to perform functions of both the breath capture module and mixing module based on a configuration of the device, as one possibility). As such, it will be appreciated that various different mechanical components may be used to facilitate the functionality of the modules, and that the following piping and instrument diagrams described herein are presented for explanatory purposes and should not be construed as limiting.

Reference will now be made to the accompanying drawings, which assist in illustrating various features of the present disclosure. The following description is presented for purposes of illustration and description. Furthermore, the description is not intended to limit the inventive aspects to the forms disclosed herein. Consequently, variations and modifications commensurate with the following teachings, and skill and knowledge of the relevant art, are within the scope of the present inventive aspects.

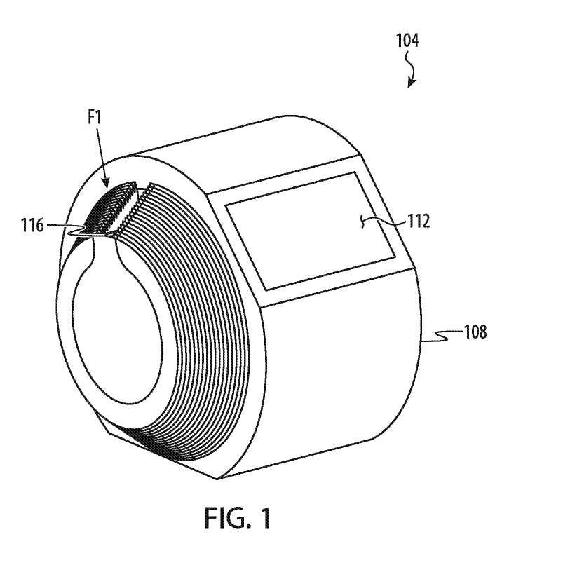

FIG. 1 depicts an analysis device 104, such as the analysis device generally discussed above and described in greater detail below. The analysis device 104 may be configured to determine an aldehyde content of a breath sample. For example, the analysis device 104 may include an unattended HPLC process and one or more systems that capture and elute aldehydes of a breath sample and detect aldehydes according to molecule size once separated by the HPLC.

The analysis device 104 may include an enclosure 108 that forms an exterior surface of the analysis device 104. As shown in FIG. 1, the enclosure 108 may form a substantially flat or planar bottom with curved or contoured surfaces extending therefrom to define a drum or cylindrical shape. It will be appreciated, however, that the enclosure 108 may form substantially any appropriate shape to define an exterior surface that conceals some or all of the various mechanical systems described herein, and thus FIG. 1 presents one possibility of such shape.

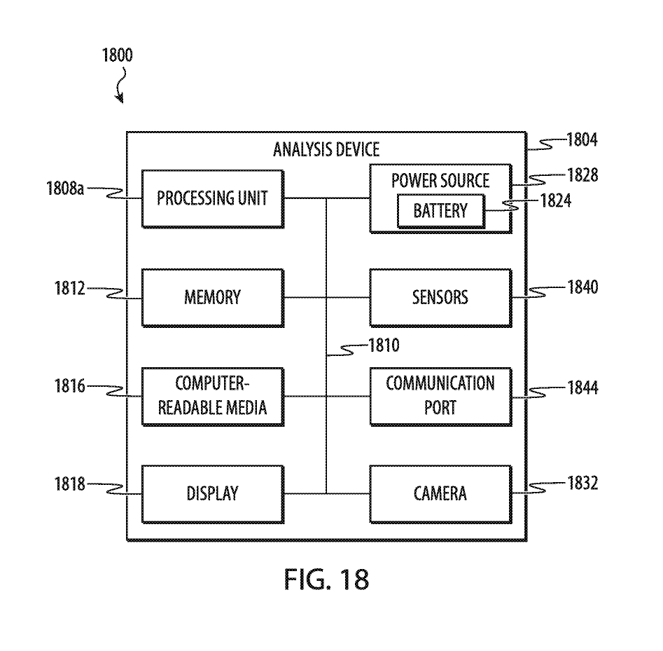

The analysis device 104 may also include a display 112. The display 112 may be at least partially positioned within the enclosure 108, such as being positioned at least partially within an opening defined in an exterior surface, and used to depict graphical objects corresponding to the determined aldehyde content or other information of the analysis device. The graphical objects may indicate a status or configuration of the analysis device 104, such as an indication of the analysis device 104 drawing a breath sample, eluting a breath sample, detecting aldehydes, and so on. Additionally or alternatively, the graphical objects may output a score or metric associated with the detected aldehydes, such as a concentration or value of one aldehyde designation relative to another aldehyde designation. In some cases, the display 112 may be a touch-sensitive display or otherwise responsive to touch or proximity inputs along the exterior surface of the enclosure 108. Accordingly, the graphical objects may prompt a user to take certain actions, such as transmitting detection results to another electronic device, providing alerts to medical personnel or other users, performing a device diagnostic, among other possibilities. This may be facilitated by a wireless or hardwired connection between the analysis device 104 and another electronic device or communication network, as described in greater detail below with respect to FIG. 18.

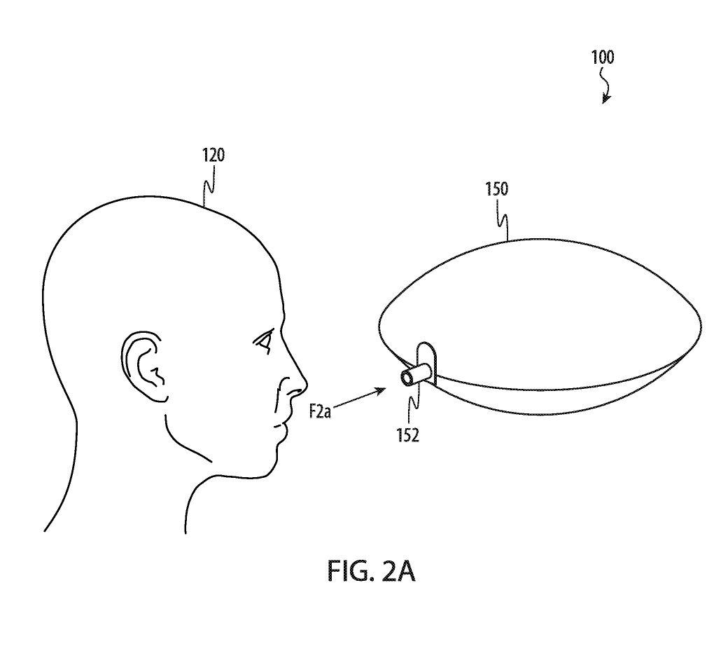

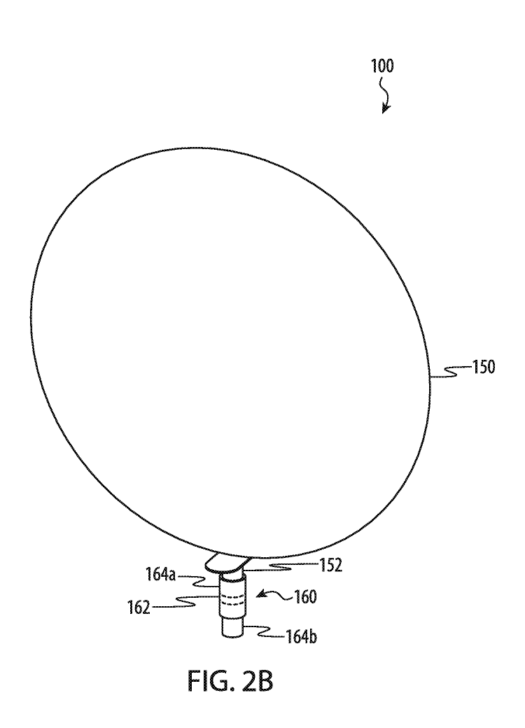

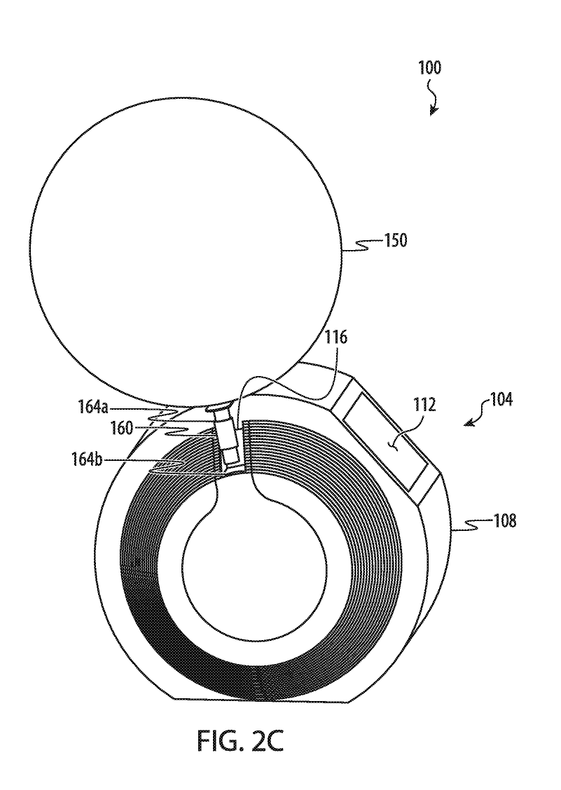

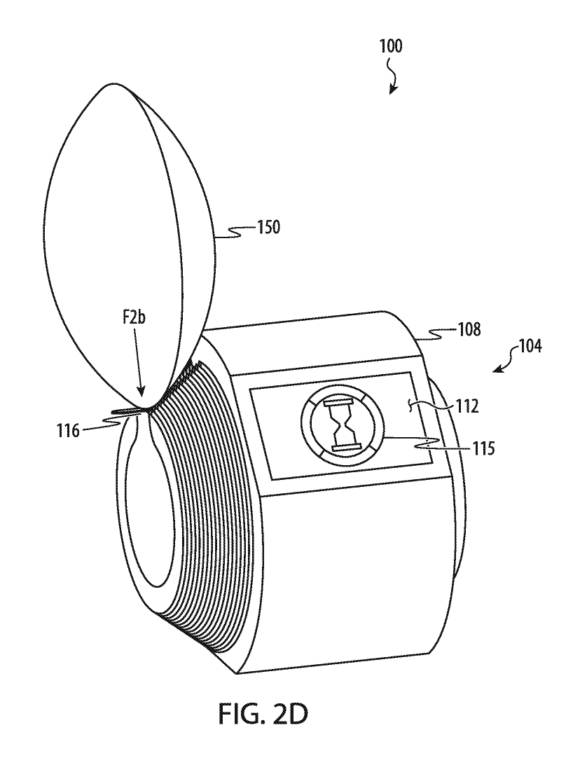

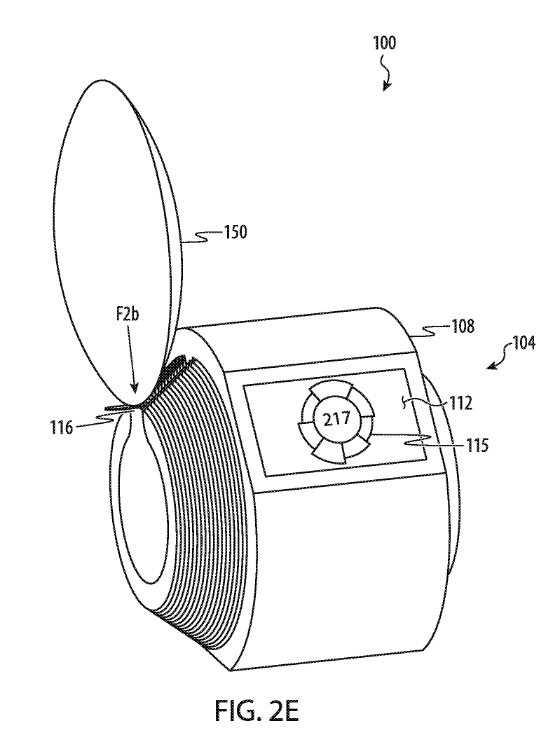

One or more openings may be defined by the enclosure 108 to facilitate receiving a breath sample and determining an aldehyde content using a group of reagents. As shown in the embodiment of FIG. 1, the enclosure may define an opening 116. The opening 116 may be positioned along an exterior side surface of the analysis device 104. The opening 116 may be configured to receive a breath sample, such as along flow path F1. In particular, and as described in greater detail below with respect to FIGS. 2A-2E, the opening 116 may be configured to receive at least a portion of a cartridge. The cartridge may have a permeable membrane used to capture aldehydes contained within a breath sample from a user or patient. Breath may thus be drawn through the opening 116 and into an interior of the analysis device 104, for example, using a pump positioned within the enclosure 108. The breath sample and retained aldehydes captured by the permeable membrane may be manipulated by various mechanical components, instruments, devices, chemical compounds, and so on concealed by the enclosure 108, in order to facilitate determination of the aldehyde content of the breath sample.

The enclosure 108 may also conceal a container or set of containers (not shown in FIG. 1) that collectively hold or serve as a temporary repository for chemical compounds, including reagents, buffers, dyes, and so on used by the analysis device 104 for aldehyde detection. The container or set of containers may be removable components that hold a quantity or volume of reagents for analysis of multiple breath samples. As such, the analysis device 104 may be used to determine an aldehyde content for multiple different breath samples before replacing containers holding the reagents. This may be beneficial, for example, when the analysis device 104 is used in a clinical setting, or other environment where multiple patient samples are analyzed consecutively. As described in greater detail below with respect to FIG. 15, the enclosure 108 may include another opening that receives a container having internal chambers that are configured to hold individual ones of the reagents. When the container is positioned within the analysis device 104, the internal chambers may each be fluidically coupled with a corresponding module or system of the analysis device 104 used to collectively determine the aldehyde content of the breath sample. Thus, the analysis device 104 may include various pumps, tubes, sensors, and so forth, to selectively dispense reagents from the container and into an internal volume of the enclosure 108.

FIGS. 2A-2E depict a breath analysis system 100, such as the breath analysis system generally discussed above and described in greater detail below. In particular, FIGS. 2A-2E depict various components of the breath analysis system 100 undergoing operations associated with aldehyde detection. The breath analysis system 100 broadly includes components that capture breath (air) from a user or patient and determine an aldehyde content or score of the breath sample. As described in the illustrative examples of FIG. 2A-2E, this may include at least a breath capture component 150, a cartridge 160, and the analysis device 104 described above with respect to FIG. 1. Other components are possible and described herein, including a cartridge configured to hold or retain one or more reagents. It will also be appreciated that FIGS. 2A-2E show one sample embodiment of breath capture and elution; other techniques and structures are possible and described herein, including embodiments where some or all of the breath capture component 150 or the cartridge 160 is included within the analysis device.

FIG. 2A depicts the breath analysis system 100 undergoing a processing step for inflating the breath capture component 150. The breath capture component 150 may be substantially any structure having an internal volume configured to retain breath received from a user or patient. As shown in FIG. 2A, the breath capture component 150 is an inflatable bag. The bag may alternate or transition from a deflated state to an inflated state when air is received through an intake 152. The intake 152 may be, form a component of, or couple with, a mouthpiece, through which a user may engage and exhale through to inflate the bag. For example, FIG. 2A shows a user 120 exhaling into the breath capture component 150 through intake 152. The exhaling user 120 may propagate a breath sample substantially along flow path F2a. As the breath sample continues along the flow path F2a, the breath capture component 150 may inflate and retain the sample for subsequent analysis.

For example, the breath capture component 150 may also include a check valve, regulator, stopper, cap, or other component to retain or temporarily seal breath within the internal volume. A check valve, for example, may allow a user to repeatedly exhale into internal volume of the breath capture component until the internal volume retains a sufficient quantity of air. In a sample embodiment, the bag may be substantially inflated when it receives at least 10 liters of air from a user. In other cases, more or less than 10 liters may be appropriate and may be tuned in order to deliver a breath sample to the analysis device 104 sufficient for detection of aldehydes.

FIG. 2B depicts the breath analysis system 100 undergoing a processing step for attaching the breath capture component 150 to the cartridge 160. As described above, the cartridge 160 may include a permeable membrane 162 (shown in phantom) positioned along a flow path within the cartridge. The permeable membrane 162 may be a silica bed or other appropriate structure that allows the breath sample to pass substantially unobstructed. The silica bed may, however, operate to capture or retain aldehydes of the breath sample when the breath sample passes through the permeable membrane. As described in greater detail below, this may allow the aldehydes to be removed from the breath sample and used to form a mobile chromatography phase for subsequent detection of aldehyde content.

To facilitate the foregoing, the cartridge 160 may include at least a first attachment region 164a and a second attachment region 164b. As shown in FIG. 2B, the first attachment region 164a may be attached to the breath capture component 150, for example, such as to the intake 152. Thus, generally, the breath sample contained within the internal volume of the breath capture component 150 may flow into the cartridge at the first attachment region 164a. At, near, or otherwise fluidically coupled to the attachment region 164, may be one or more check valves, regulators, or the like to prevent breath or other fluid from traversing into the breath capture component 150 from the cartridge 160.

In a sample embodiment, the second attachment region 164b may be used to fluidically couple the cartridge 160 with the analysis device 104. The breath sample may flow through or exit the cartridge 160 from the second attachment region 164b and into the analysis device 104. The second attachment region 164b is shown in FIG. 2B as being opposite an elongated body of the cartridge 160; however, this is not required. In other cases, the second attachment region 164b may be positioned along a side or another end of the cartridge 160. The permeable membrane 162 may generally be positioned between the first attachment region 164a and the second attachment region 164b, so that the breath sample may flow through the permeable membrane 162 as it flows from the first attachment region 164a to the second attachment region 162b. As described in greater detail below, the second attachment region 164b may include or otherwise be fluidically coupled with multiple inlet and/or outlet structures of the analysis device 104, such as, the structure may be used to pull air through the permeable membrane 162, elute aldehydes from the permeable membrane 162, purge or clean internal components of the analysis device 104, among other possibilities.

FIG. 2C depicts the breath analysis system 100 undergoing a processing step for attaching the cartridge 160 to the analysis device 104. As shown in FIG. 2C, the cartridge 160 may be at least partially positioned in or received by the opening 116 defined along an exterior of the enclosure 108. For example, the second attachment region 164b of the cartridge 160 may be advanced into the opening 116 and fluidically coupled with one or more inlet and/or outlet structures of the analysis device 104. In other cases, the cartridge 160 may be fluidically coupled with the analysis device 104 using other openings or features in the analysis device 104, including being positioned fully within the enclosure 108, thereby concealing or partially concealing the cartridge 160 and/or the breath capture component 150. The cartridge 160 may be attached to the analysis device 104 while having the breath capture component 150 attached to the first attachment region 164a. Alternatively, the cartridge 160 may be attached to the analysis device 104 in a configuration in which the first attachment region 164a may be substantially uncoupled from the breath capture component 150. The display 112 may optionally indicate coupling of the cartridge 160 to the analysis device 104 and/or convey information to a user regarding a prompt or alert for initiation of aldehyde detection of the analysis device 104.

FIG. 2D depicts the breath analysis system 100 in a configuration corresponding to an initiation of aldehyde detection of the breath sample. For example, the analysis device 104 may initiate a process for detecting an aldehyde content of the breath sample upon coupling of the cartridge 160 (not shown in FIG. 2D) in the opening 116 or other component or feature of the analysis device 104. This may be initiated upon a user command or input (such as that received at the display 112), or may occur upon a detection of the cartridge 160 and the analysis device 104 (or after a delay, which may be programmable).

As shown in FIG. 2D, the analysis device 104 may pull the breath sample from the breath capture component 150 along a flow path F2b. The flow path F2b may extend from the breath capture component 150, through the cartridge 160, and into the analysis device 104. The analysis device 104, for example, may include one or more vacuum pumps, or other biasing elements, that draw the breath along the flow path F2b. The flow path F2b may extend through the permeable membrane 162 (not shown in FIG. 2D) that is positioned within the cartridge 160. As the analysis device 104 draws the breath sample along the flow path F2b, aldehydes of the breath sample may be trapped, captured, or otherwise retained by the permeable membrane 162. The captured aldehydes may be representative of the quantity of aldehydes in the breath sample. As such, the retained aldehydes may be eluted and analyzed in order to determine an aldehyde content of the breath sample, according to the various techniques described herein.

FIG. 2D also shows the display 112 having a graphical output 115. The graphical output 115 may correspond to the configuration of the analysis device 104, such as a configuration in which air is drawn through the permeable membrane 162. The graphical output 115 may thus be updatable to convey information associated with other configurations or operations of the analysis device 104, such as configurations associated with eluting retained aldehydes from the permeable membrane 162, mixing the elution with reagents, including catalysts and dyes, performing an HPLC process, among other configurations.

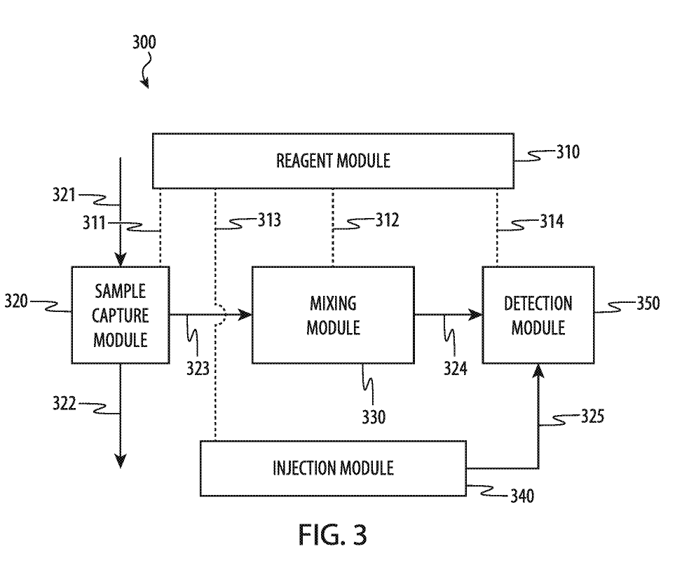

FIG. 2E depicts the breath analysis system 100 upon completion of aldehyde detection of the breath sample. Subsequent to the breath sample being drawn through the permeable membrane 162, described above with respect to FIG. 2D, the analysis device 104 may perform various different operations to detect the aldehyde content of the breath sample, described below with respect to FIGS. 3-14. This may include, among other operations, forming a mobile chromatography phase from an elution of retained aldehydes, performing an HPLC process, and determining an aldehyde content based on the fluorescence of separated particles. Each of these and other processes of the analysis device 104 may be integrated and streamlined, requiring little, if any, input from a user. Upon completion, the analysis device 104 may convey information to the user corresponding to the aldehyde content of the breath sample. For example, as shown in FIG. 2E, the display 112 may include a graphical output 115 that may include one or more symbols, pictures, glyphs, numbers, or the like that represent information concerning the aldehyde content of the breath sample. In one embodiment, the graphical output 115 may be a chart (such as a circular or pie chart) having bars or areas corresponding to a value (quantity) of certain aldehyde groups of a given size or designation relative to other aldehyde groups. For example, the graphical output 115 may have an individual area corresponding to a relative value of each of the aldehydes designated C4-C10. This may allow a user to readily understand the quantity of a particular aldehyde, and discern information relating to the oxidative stress present in the breath sample, for example, by reference to the relative quantities of other aldehydes. In some cases, the graphical output 115 may also include an aldehyde score or metric, which may be a composite or derived output based on the relative quantities of the distinct aldehydes detected by the analysis device 104.

In the embodiment of FIG. 2E, the cartridge 160 (and associated breath capture component 150) is shown attached to the analysis device 104. Subsequent to (or during) the detection of aldehydes by the analysis device 104, the cartridge 160 may be removed from the analysis device 104. In this regard, the analysis device 104 may be further configured to receive another cartridge (having another associated breath capture component) for analysis of further breath samples. For example, in a configuration, internal components of the analysis device 104 may be sanitized, purged, primed and so on in order to detect aldehydes in additional breath samples. Thus, the analysis device 104 may be configured for repeated detection of aldehydes for multiple breath samples, thereby enhancing the adaptability of the analysis device 104 in clinical settings. For example, the analysis device 104, and breath analysis system 100 more generally, may be used in a clinical setting in which breath samples from multiple patients are analyzed for aldehyde content with little to no input from a trained operator.

FIG. 3 depicts a functional diagram of an analysis device 300. The analysis device 300 may be substantially analogous to the analysis device 104 described above with respect to FIGS. 1-2E. Accordingly, the analysis device 300 may be configured to detect an aldehyde content of a patient breath sample. For example, the analysis device 300 may form a mobile chromatography phase having aldehydes captured from a patient breath sample. The aldehydes may be separated through an HPLC process and subsequently detected by molecule size using a detection assembly.

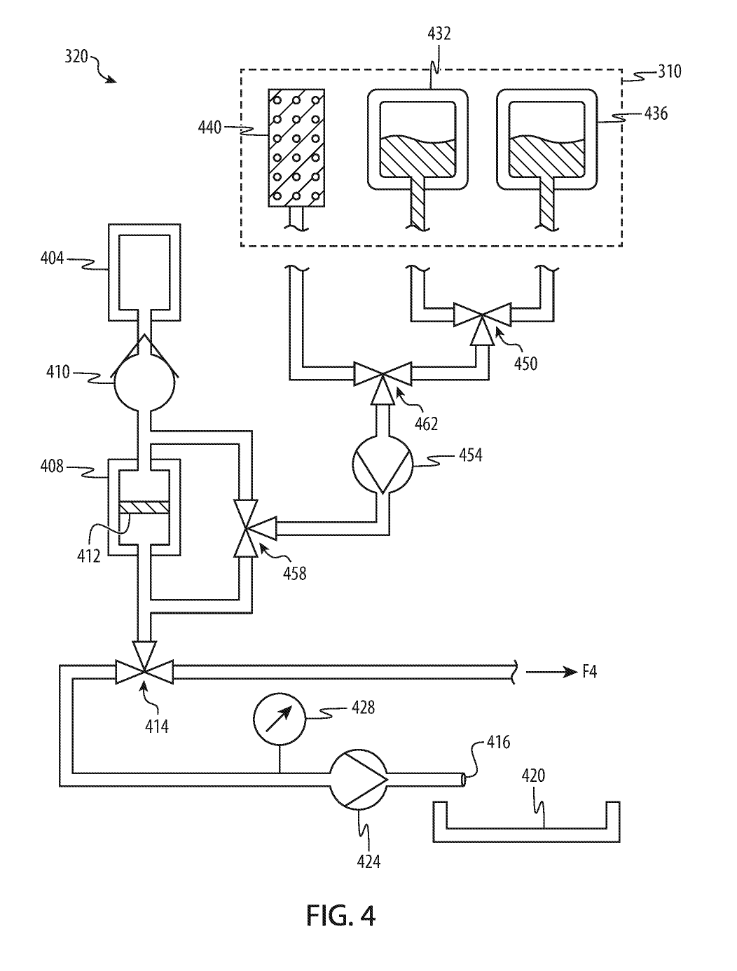

The analysis device 300 may include various modules or collections of mechanical components, instruments, and so on that collectively operate to perform the functions described herein. For example, and as shown in FIG. 3, the analysis device 300 may include at least a reagent module 310, a sample capture module 320, a mixing module 330, an injection module 340, and a detection module 350. Rather that define discrete or separated mechanical components and instruments, it will be appreciated that the modules may use common or overlapping components and instruments to perform the functions herein. For example, a given pump, value, or other element may be used to perform a function of the sample capture module 320 in a first configuration, a function of the mixing module 330 in a second configuration, a function of the detection module 350 in a third configuration, and so forth. Accordingly, the individual modules discussed with respect to FIG. 3 are used to facilitate an understanding of the analysis device 300, and are not meant as limiting or demarcating specific mechanical components or instruments as performing an isolated function. As such, the compounds described below with respect to FIGS. 4-12I are presented as one possible implementation of the modules described with respect to FIG. 3, and are not meant as limiting.

The reagent module 310 may include some or all of the chemical compounds used by the analysis device 300 for detection of the aldehyde content of a breath sample. For example, the reagent container may include MeOH 40%, MeOH 100%, a buffer, a calibrant, a catalyst, a dye, and/or other chemical compounds that may be selectively used by various other modules of the analysis device 300 in order to facilitate aldehyde detection of the breath sample. The reagent module 310 may include a quantity of each of these, or other chemical compounds sufficient for the analysis device 300 to detect aldehydes of multiple successive breath samples. Thus, while the reagent module 310 may be a removable component coupled within the analysis device 300, it may be used across multiple analyses of the analysis device 300. The reagent module 310 may also include various other components that may facilitate aldehyde detection of the analysis device 300, including one or more filters (for filtered air intake) and a waste receptacle, among other components and features. These too may be used for multiple successive breath sample analyses and may be tuned or calibrated according to a limiting quantity of one or more of the chemical compounds. For example, the waste receptacle may be substantially full when one or more of the chemical compounds is substantially consumed by a predetermined amount of breath sample analyses.

To facilitate the foregoing, the reagent module 310 may be fluidically coupled with each of the other modules of the analysis device 300. As shown in the non-limiting example of FIG. 3, the reagent module 310 may be fluidically coupled with each of the sample capture module 320, the mixing module 330, the injection module 340, and the detection module 350. In particular, an illustrative reagent path 311 is shown fluidically coupling the reagent module 310 and the sample capture module 320, an illustrative reagent path 312 is shown fluidically coupling the reagent module 310 and the mixing module 330, an illustrative reagent path 313 is shown fluidically coupling the reagent module 310 and the injection module 340, and an illustrative reagent path 314 is shown fluidically coupling the reagent module 310 and the detection module 350. It will be appreciated that rather than depict a particular flow path or direction, or particular chemical compound or fluid, the illustrative reagent paths are shown to generally depict fluidic coupling. Each of the reagent paths may include connections for multiple different chemical compounds (or air intake exhaust, etc.) to and/or from the respective modules and which may operate at different times based on a given configuration of the analysis device 300. Accordingly, each of the modules described herein may include, or be fluidically coupled with, pumps, valves, instruments, and so on that may selectively dispense chemical compounds from the reagent module 310 when used to facilitate the performance of a particular operation of the analysis device 300.

FIG. 3 also shows the sample capture module 320. The sample capture module 320 may be used to capture aldehydes from a breath sample (e.g., on a silica bed) and elute the captured aldehydes. The elution may be transferred to the mixing module 330, as described below, to form a mobile chromatography phase having aldehydes from the breath sample.

Broadly, the sample capture module 320 may receive a breath sample at flow 321. The flow 321 may be received from, for example, a breath capture component (breath capture component 150 of FIG. 2A) or other device (or the patient) and contain aldehydes. The sample capture module 320 may initiate the flow 321 using a vacuum pump or other biasing component that draws the breath sample through a permeable membrane (silica bed). Accordingly, the sample capture module 320 may output the breath sample at flow 322. When the breath sample exits the sample capture module 320 at the flow 322, it may be free of a representative sample of aldehydes (including free of substantially all aldehydes), for example, which may have been captured by the permeable membrane of the sample capture module 320.

The sample capture module 320 may be further configured to form an elution having the aldehydes captured by the permeable membrane. For example, the sample capture module 320 may initiate a flow of reagents or other chemical compounds from the reagent module 310 (e.g., using the reagent path 311) that elutes the permeable membrane. For example, a MeOH 40% reagent may be used to substantially dissolve the reagents (or a representative sample thereof) from the permeable membrane in order to form an elution (liquid) containing aldehydes of the breath sample. This elution having the aldehydes of the breath sample may be output to the mixing module 330 at flow 323.

The mixing module 330 may be configured to receive the elution at flow 323 and form a mobile (liquid) chromatography phase. Broadly, to facilitate the foregoing, the mixing module 330 may obtain multiple, different chemical compounds from the reagent module 310 along the reagent path 312. This may include, without limitation, a calibrant, a catalyst, and a dye. The calibrant may be a standardized solution having a known aldehyde content of a particular molecule size or designation. This known aldehyde content may be used as a baseline or reference point for determining the relative aldehyde content of other particular aldehyde groups of designations contained within the breath sample. The catalyst may be used to facilitate a chemical reaction that forms the mobile chromatography phase from the elution and other chemical compounds from the reagent module 310. The dye may be a fluorescent compound responsive to radiation, such as from a laser. The dye may attach to aldehydes within the mixing module 330 and used as an indicator (marker) of a presence of aldehydes once separated within the detection module 350.

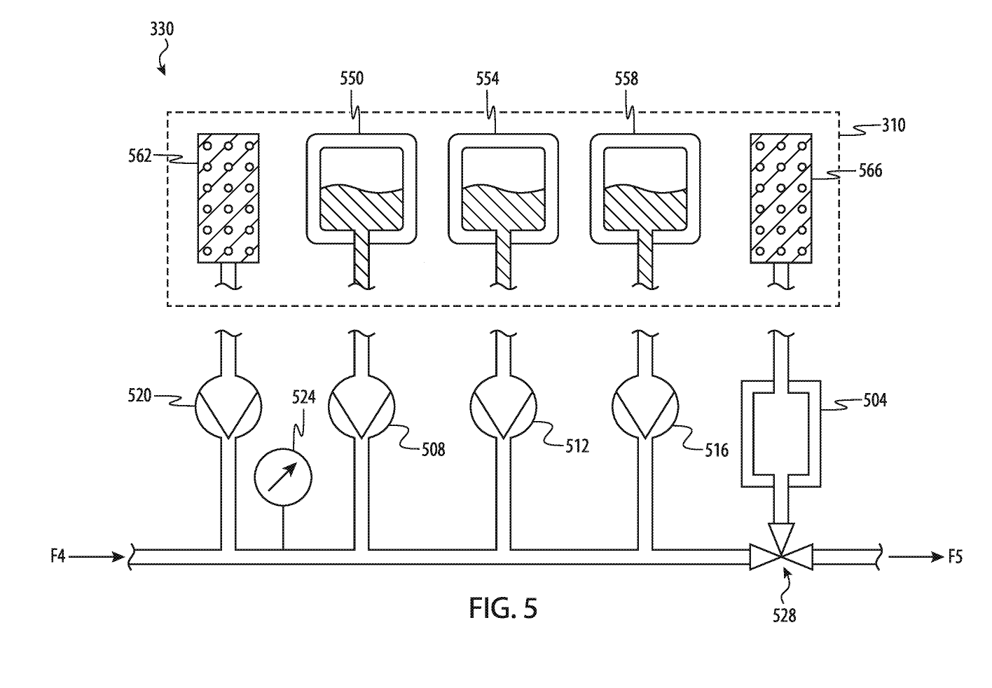

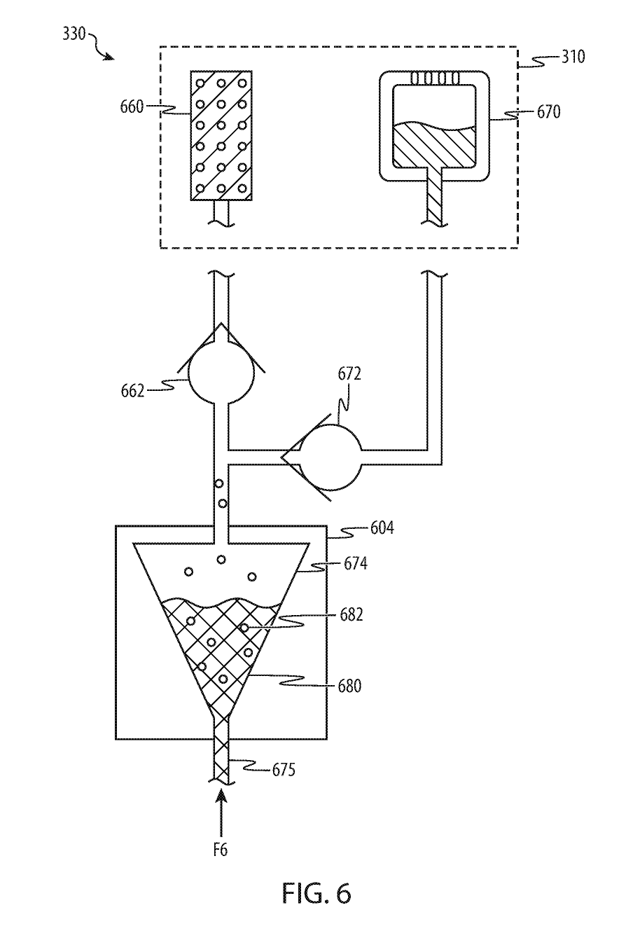

Each of the foregoing chemical compounds from the reagent module 310 may be mixed with the elution within a mixing volume of the mixing module 330, described in greater detail below with respect to FIGS. 5 and 6. The chemical compounds may be added to the elution in any appropriate manner, including sequentially or in combination, and may be added prior to or during the filling of the mixing volume with the elution. The mixing module 330 may mix the elution with the chemical compounds using air agitation or bubbles that percolate through the contents of the mixing volume. The bubbles may be drawn from the atmosphere through a filter of the reagent module 310. For example, when the mixing volume is filled, the mixing module 330 may initiate air flow along one or both of the reagent paths 311 or 312 that draws air through a filter contained within the reagent module 310 and into the mixing volume. It will be appreciated, however, that in some cases the filter may be a separate component of the analysis device 300 and need not necessarily be associated with the reagent module 310. Alternatively, the filter may be optional. Further, in other cases, the elution and chemical compounds may be mixed by other techniques, including mechanical agitation, thermal mixing, among other techniques. Notwithstanding, upon completion of the mixing, the mixing module 330 is configured to output the mobile chromatography phase formed within the mixing volume at flow path 324. As described below, the mobile chromatography phase of the flow path 324 is advanced through the detection module 350 by an output of the injection module 340, whereat the aldehyde content may be detected using the HPLC and optical detection. Separated from, and parallel to, the mixing module 330, the injection module 340 may therefore be configured to form a pressurized flow that is used to advance the mobile chromatography phase through the detection module 350. The pressurized flow may include at least a reagent and a buffer received from the reagent module 310 along the reagent path 313. The buffer may control a concentration of the reagent, which may be variable based on a predefined gradient ramp. For example, and as described in greater detail below, the concentration of the reagent may increase as the mobile chromatography phase is advanced through the detection module. This increase in concentration may allow for progressively larger aldehyde molecules to propagate through a column or other separation instrument or structure of the detection module 350, effectively separating the aldehydes of the mobile chromatography phase by molecule size.

To facilitate the foregoing, the injection module 340 may include two high pressure pumps, operating in parallel, that each draw a respective one of the reagent and the buffer from the reagent module 310. The output of each of such parallel high pressure pumps may be combined at a static mixing tee and output to the detection module 350 along flow 325. Alternatively, the reagent and buffer may be combined according to selectively controlled ratios before a single high pressure pump that produces the flow 325. In either case, the injection module 340 may also be configured to monitor a status of the high pressure pumps (e.g., using an in line flow meter, or other instrument) in order to detect a cavitation of depressurization event. As explained in greater detail below, upon detection of such depressurization, the analysis device 300 may trigger a diagnostic or other configuration to prime the pumps or otherwise repressurize the flow 325.

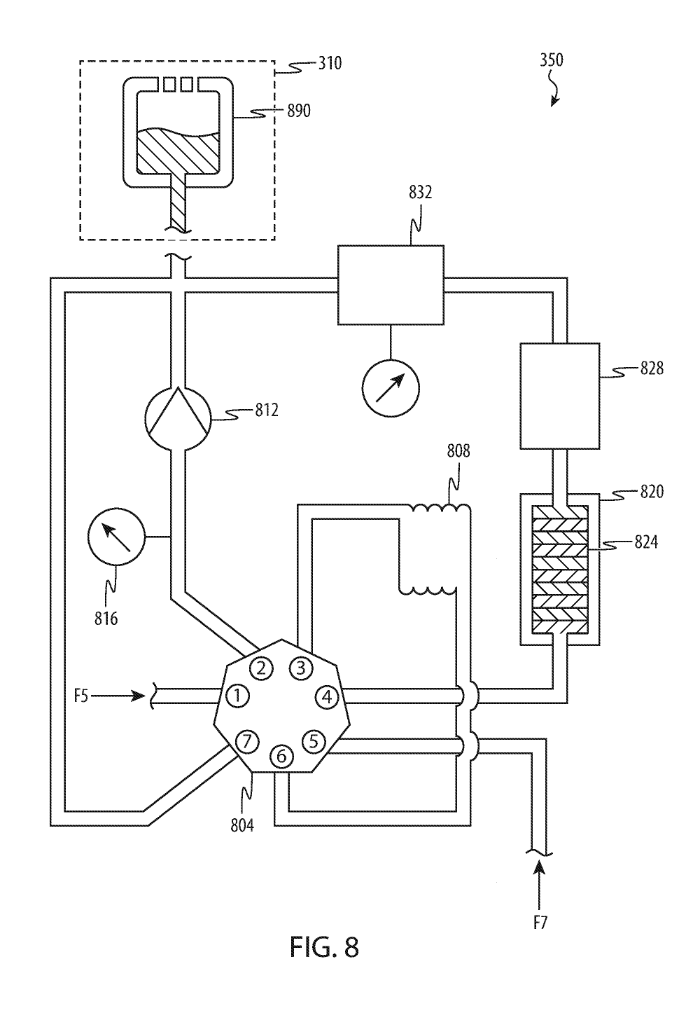

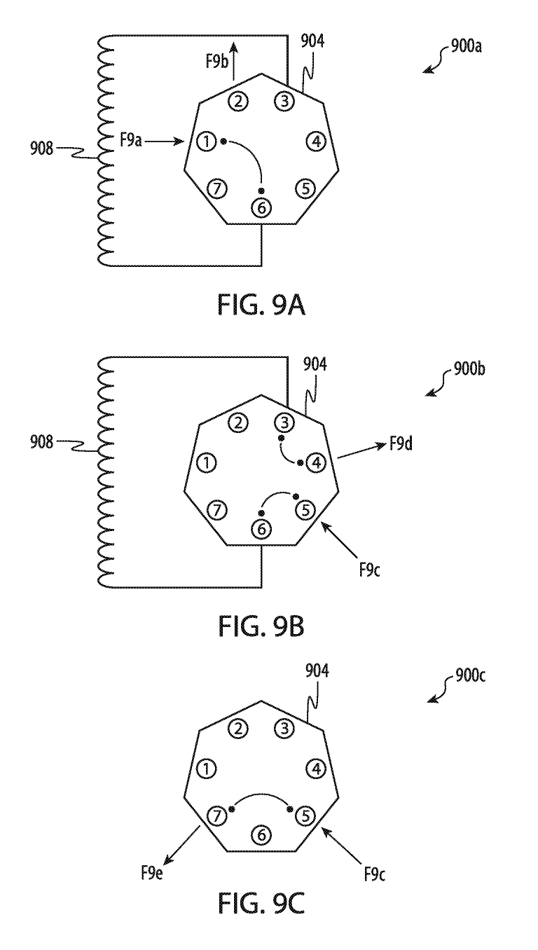

The detection module 350 may use the flow 324 from the mixing module 330 and the flow 325 from the injection module 340 to detect an aldehyde content of a patient breath sample. In a first configuration, the detection module 350 may be configured to load a sample loop with the mobile chromatography phase output from the mixing module 330 at the flow 324. For example, one or more pumps may draw the mobile chromatography solution from the mixing volume into a sample loop. The sample loop may be fluidically coupled with a control valve, such as a multi-position, multi-port value, described in greater detail below with respect to FIGS. 8-9C. Accordingly, in a second configuration, the detection module 350 may be configured to rotate or reposition the sample loop (using the control valve) in order to fluidically couple the sample loop with the pressurized output of the injection module 340 using the flow 325.

Upon coupling with the pressurized output of the injection module 340, the detection module 350 may be configured to separate aldehydes of the mobile chromatography phase according to molecule size. For example, the detection module 350 may include a column or other separation structure or device having a high-density silica bed (stationary chromatography phase). The high-density silica bed may generally impede the propagation of aldehydes therethrough. However, with the aid of the pressurized output from the injection module 340, the mobile chromatography phase may be advanced through the column and separated according to molecule size or designation (e.g., aldehyde C4, C5, C6, and so on).

For example, the advancement of aldehydes through the column may at least partially depend on the chemical composition of the pressurized output of the injection module 340. For example, the pressurized output of the injection module 340 may include a reagent having a concentration controlled by a buffer and an initial reagent concentration that allows the smallest of the aldehyde groups of designations to progress through the column. This concentration, for example, may correspond to a concentration of reagent used to elute the aldehydes from the permeable membrane (e.g., MeOH 40%); however, other concentrations and reagents may be used. The concentration of the reagent may be increased over time according to a gradient ramp (e.g., by dynamically altering a reagent/buffer ratio). Accordingly, the gradient ramp may be a curve that defines the concentration of the reagent over a duration of the HPLC process. Generally, this concentration increases at a rate that allows the concentration of the reagent to progress from the initial MeOH 40% to a final concentration of at or near MeOH 100%. The rate, however, may be variable or otherwise non-constant as may be appropriate to facilitate the separation of aldehydes within the column. And as the concentration of reagent increases, progressively larger aldehydes may propagate through the column.

Aldehyde groups of certain sizes or designation may thus pass through the column in groups (slugs, clusters, etc.) when the concentration of reagent in the pressurized output of the injection module reaches a designated value. In this regard, the gradient ramp may be controlled to allow the various aldehyde groups to pass through the column separated from one another (in bands) to aid in detection of relative aldehyde content at an output of the column. In this manner, when aldehydes are detected at the output of the column, a processing unit of the analysis device 300 (or another electronic device) may associate the detected aldehydes with a certain aldehyde designation (e.g., C4, C5, C6, etc.) based on an anticipated propagation time of the aldehyde group through the column, as determined by the gradient ramp and increasing concentration of reagent in the pressurized output from the injection module 340.

The detection module may be configured to measure an output of the column to detect aldehydes. In one embodiment, the detection module may be configured to optically detect aldehydes. In particular, the output of the column may be hit by an excitation source (such as radiation from a laser). As described above, the fluorescent may be attached to a phosphorescent dye. As such, when passed through a path of the excitation source, the dye may fluoresce, and thus indicate a presence of aldehydes. The detection module 350 may therefore be configured to detect an increase in brightness of the output of the column. As one possibility, the output of the column may extend between the excitation source (laser) and a detector. The detector may include, or be coupled with, a band-pass or other filter, thereby allowing the detector to register an optical signal in response to fluorescence of the dye. In some cases, the optical signal may correspond to a value of the increase in brightness, and thus be used to determine a relative quantity of a detected aldehyde (e.g., by comparing a brightness value for a given aldehyde group with other brightness values for other aldehyde groups. This signal from the detector may thus be processed to determine the foregoing and communicate to a user a determined aldehyde content of the breath sample (e.g., using one or more graphical outputs of a display). As previously mentioned, some embodiments may employ different modes of detection and/or separation of aldehydes, including other chemical or physical properties, such as size, shape, hydrophobicity, hydrophilicity, charge, polarity, and so on.

As shown in FIG. 3, the detection module 350 may also be fluidically coupled with the reagent module 310, for example, through reagent path 314. For example, the detection module 350 may output a waste stream to the reagent module 310. This may include the mobile chromatography phase output from the column, along with any other chemical compounds or gasses that result from the operation of the detection module 350.

FIG. 4 depicts sample components and instruments that may cooperate to implement and perform one or more functions of the sample capture module 320. The various components and instruments described herein with respect to FIG. 4 are presented to facilitate an understanding of the sample capture module 320 and are not meant as limiting. To the contrary, the described embodiment of FIG. 4 is intended to cover alternatives, modifications, and equivalents of the sample capture module 320 consistent with the teachings herein.

FIG. 4 shows a breath capture component 404. The breath capture component 404 may be substantially analogous to the breath capture component 150 described above with respect to FIGS. 2A-2E. As such, the breath capture component 404 may be configured to hold a breath sample. The breath capture component 404 may be coupled with a cartridge 408. The cartridge 408 may be substantially analogous to the cartridge 160 described above with respect to FIGS. 2A-2E. As such, the cartridge 408 may include a permeable membrane 412 positioned along an internal flow path. The permeable membrane 412 may be a silica bed configured to retain aldehydes when the breath sample passes through the cartridge 408. A directional valve 410 (check valve, regulator, or the like) may be positioned along a flow path between the breath capture component 404 and the cartridge 408. The directional valve 410 may substantially prevent flow into the breath capture component 404, including the flow of reagents, solvents, and/or other chemical compounds that may be used to flush or propagate through the cartridge 408 subsequent to aldehyde capture. A multi-position valve 414 (three-way value) may be positioned along a flow path extending from an outlet of the cartridge 408. The multi-position valve 414 may be used to alternate a flow path at the outlet of the cartridge 408 between an exhaust (and associated drip pan) of the analysis device 300 and the mixing module 330.

For example, in a first configuration, the multi-position valve 414 may be configured to route flow from the outlet of the cartridge 408 toward an exhaust 416. When the multi-position valve 414 is in the first configuration, flow from the outlet of the cartridge 408 may be substantially blocked from proceeding to the mixing module 330. The exhaust 416 may be open to atmosphere or otherwise to allow fluid (air) to exit the sample capture module 320. The exhaust 416 may be coupled with or positioned near a pan 420. The pan 420 may be a drip pan that is used to collect liquids emitted at the exhaust 416. The pan 420 may collect, for example, water or other fluids, present in a breath sample.

A pump 424 may be used to draw the breath sample held within the breath capture component 404 through the cartridge 408 and toward the exhaust 416. The pump 424 may be a vacuum pump or other suitable pump that may deflate the breath capture component 404 and pull the breath sample through the permeable membrane 412. The pump 424 may have a variable flow rate controlled by the analysis device 300. In one embodiment, the pump 424 may have a flow rate of 3 L/min.; however, this may be adjusted up or down. A flow instrument 428 may be fluidically coupled with the pump 424. The flow instrument 428 may be an inline flow meter, but other instruments are possible as well, including a pressure gauge that detects a value associated with an output of the cartridge 408. The flow instrument 428 may be used to monitor propagation of the breath sample through the permeable membrane 412, including flow rate. As such, the pump 424 may, in certain embodiments, operate at least partially based on an output or signal from the flow instrument 428. For example, when the flow instrument 428 detects a certain value (such as that corresponding to a substantial evacuation of the breath sample from the breath capture component 404), the pump 424 may cease operation. This may also cause the sample capture module 320 to initiate elution of aldehydes from the permeable membrane 412, as described herein.

In a second configuration, the multi-position valve 414 may direct flow from the outlet of the cartridge 408 to the mixing module 330 (not shown in FIG. 4) along flow path F4 which may be received by the mixing module 330. This may occur subsequent to the operation of the pump 424 that pulls the breath sample from the breath capture component 404 to the exhaust 416 (for aldehyde capture at the permeable membrane 412). When the multi-position valve 414 is in the second configuration, flow from the outlet of the cartridge 408 may be substantially blocked from proceeding to the exhaust 416. Accordingly, this may allow the sample capture module 320 to propagate an elution to the mixing module 330 for further processing.

To facilitate the foregoing, the sample capture module 320 may be configured to selectively dispense reagents from the reagent module 310 that may facilitate in forming an elution. As described above with respect to FIG. 3, the reagent module 310 may include multiple chemical compounds, filters, receptacles, and/or other compounds or structures. Shown in FIG. 4 are possible chemical compounds and filters that may be fluidically coupled with the sample capture module 320. It will be appreciated, however, that the sample capture module 320 may be fluidically coupled with more or fewer items of the reagent module 310. Further, reagent module 310 may include additional chemical compounds and structures, for example, such as those described below with respect to FIGS. 5-8.

In the embodiment of FIG. 4, the sample capture module 320 is fluidically coupled with a first sample capture reagent 432, a second sample capture reagent 436, and a sample capture filter 440. The first sample capture reagent 432 may be MeOH 40%, the second sample capture reagent 436 may be MeOH 100%, and the sample capture filter 440 may be an air filter, such as a carbon-based filter; however, other chemical compounds and structures are possible. Broadly, the first sample capture reagent 432 may be used to elute retained aldehydes from the permeable membrane 412. The second sample capture reagent 436 may be used to clean or sanitize internal components of the sample capture module 320 (and other fluidically connected module), for example, which may occur between breath analyses. The sample capture filter 440 may be used as an air intake for air agitation by the mixing module 330, air drying of internal components of the sample capture module 320, among other functions.

One or more valves, pumps, and/or other components or instruments of the sample capture module 320 may operate to selectively dispense the chemical compounds from the reagent module 310. As shown in FIG. 4, the sample capture module 320 may include a multi-position valve 450. The multi-position valve 450 may be used to establish a flow of reagent into the sample capture module 320 that alternates between the first sample capture reagent 432 and the second sample capture reagent 436. For example, when the multi-position valve 450 is in a first configuration, the first sample capture reagent 432 may flow into the sample capture module 320. Conversely, when the multi-position valve 450 is in a second configuration, the second sample capture reagent 436 may flow into the sample capture module 320.

Flow of the first sample capture reagent 432 and the second sample capture reagent 436 may be initiated or controlled by a pump 454. The pump 454 may be a fixed volume (displacement) pump that is configured to dispense a predefined volume of the respect reagents into the sample capture module 320 (e.g., as may be calibrated in micro liters). The pump 454 may be fluidically coupled with the cartridge 412 using a multi-position valve 458. The multi-position valve 458 may be configured to alternate an output of the pump 454 between a flow path that extends through the cartridge 408 (and through the permeable membrane 412) and another flow path that bypasses the cartridge 468 and proceeds to the exhaust 416 and/or the mixing module 330 (e.g., based on a configuration of the multi-position valve 414).

The pump 454 may also be used to draw air into the sample capture module 320. As shown in FIG. 4, the pump 454 may be fluidically coupled to the sample capture module 320 (and associated sample capture filter 440) using a multi-position valve 462. The multi-position valve 462 may be used to alternate an intake of the pump 454 between air from the sample capture filter 440 and one or both of the first sample capture reagent 432 and the second sample capture reagent 436. In this regard, when the multi-position valve 462 is in a first configuration, the pump 454 may operate to pump substantially liquid chemical compounds from the reagent module 310 and into the sample capture module 320. Conversely, when the multi-position valve 462 is in a second configuration, the pump 454 may operate to pump substantially gaseous fluids (e.g., filtered air) from, or as filtered through, the reagent module 310 and into the sample capture module 320.

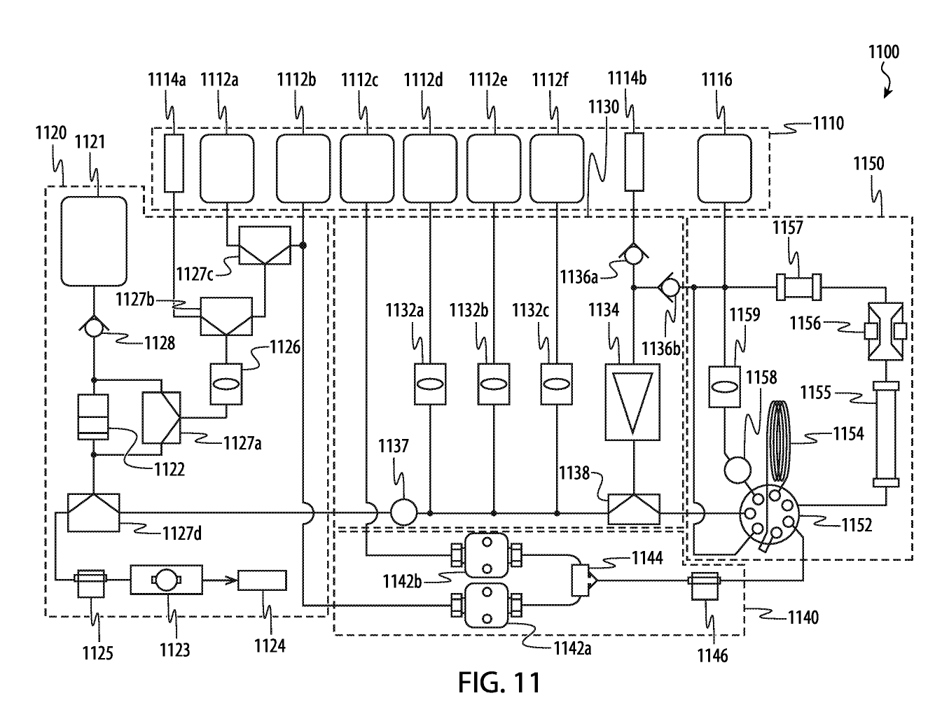

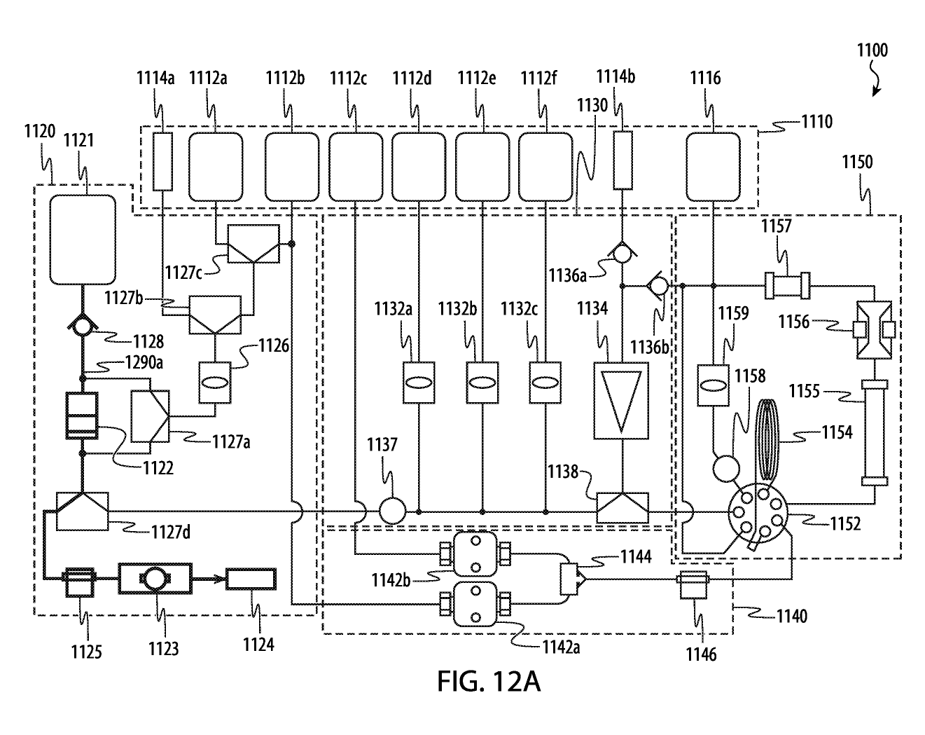

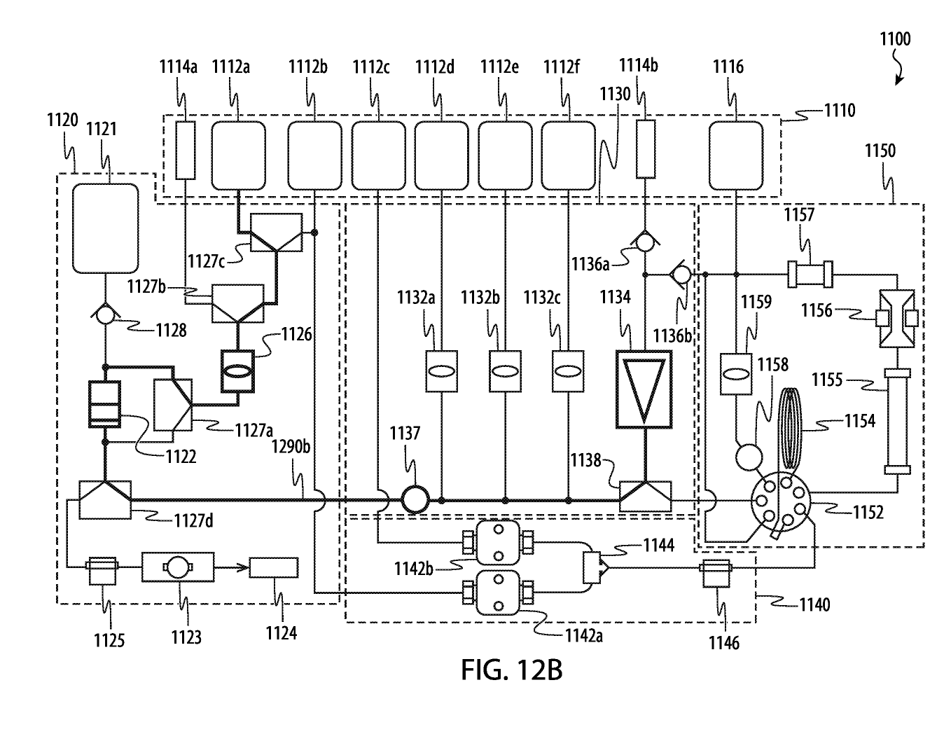

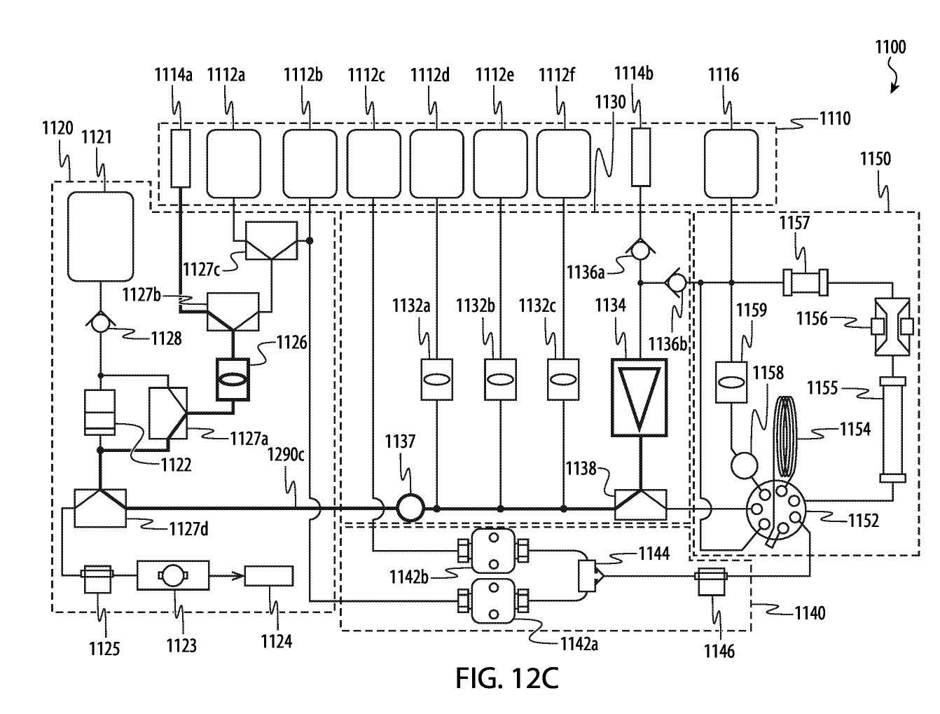

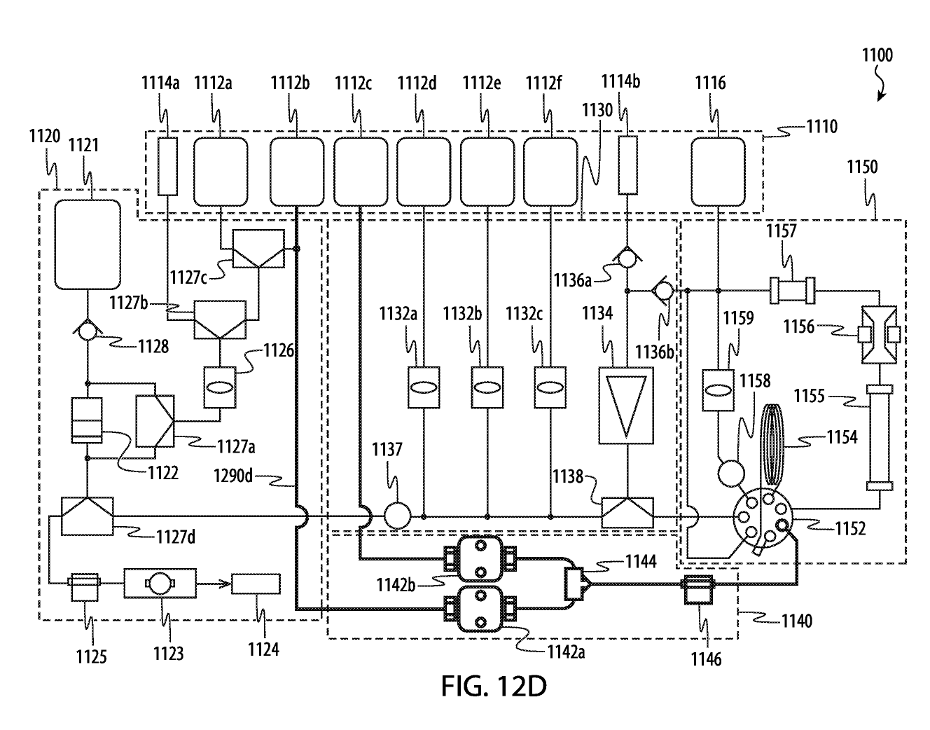

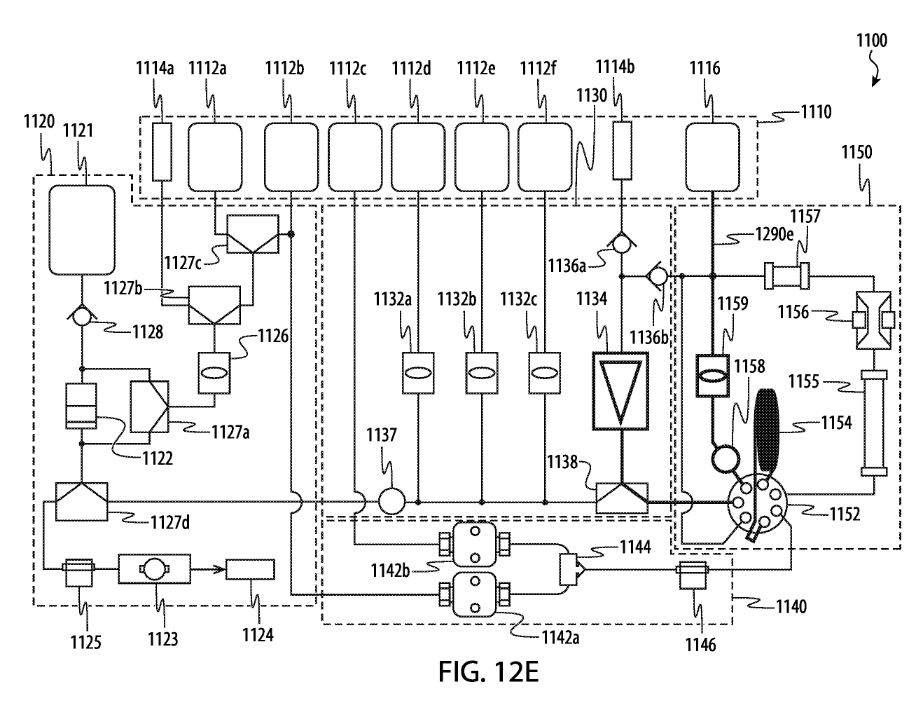

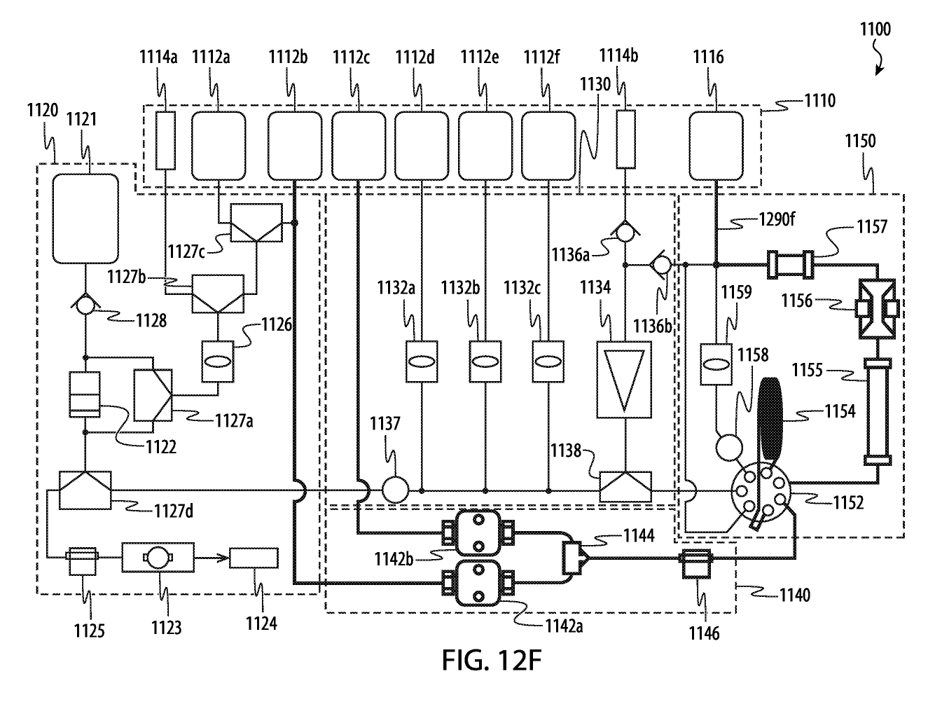

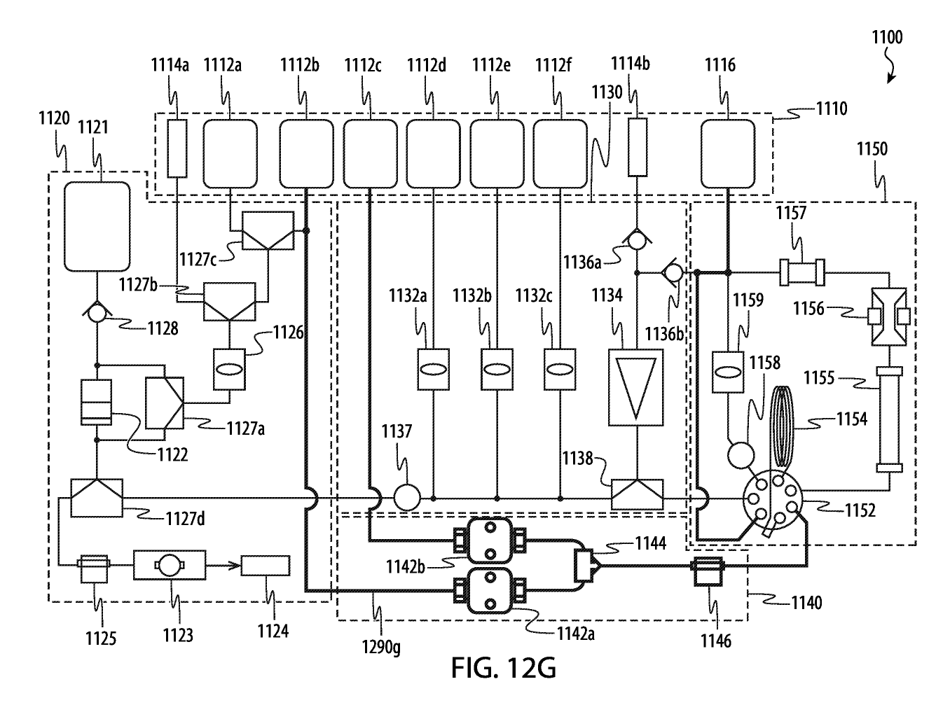

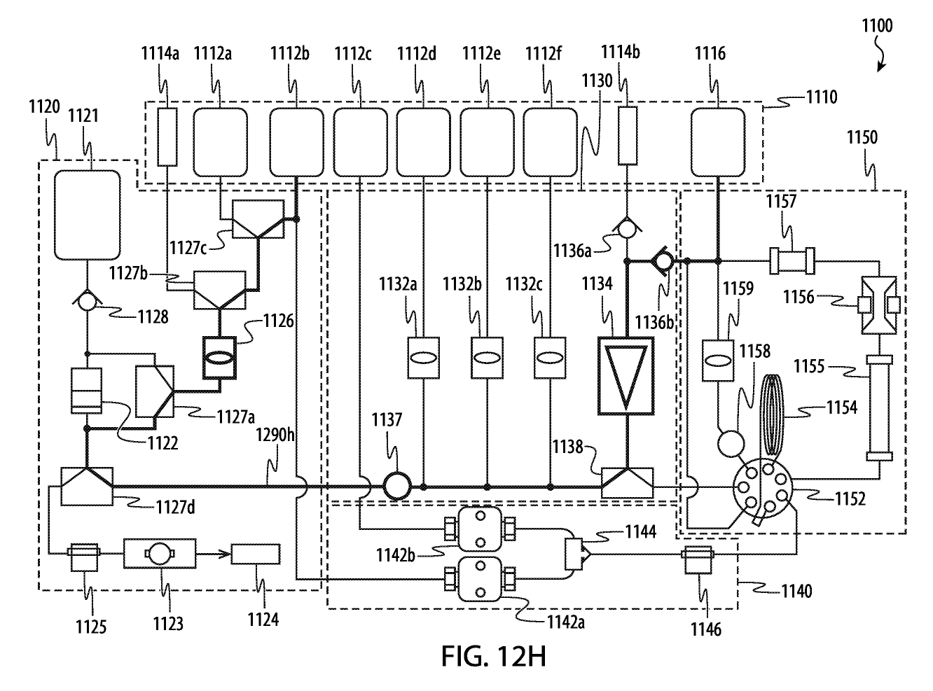

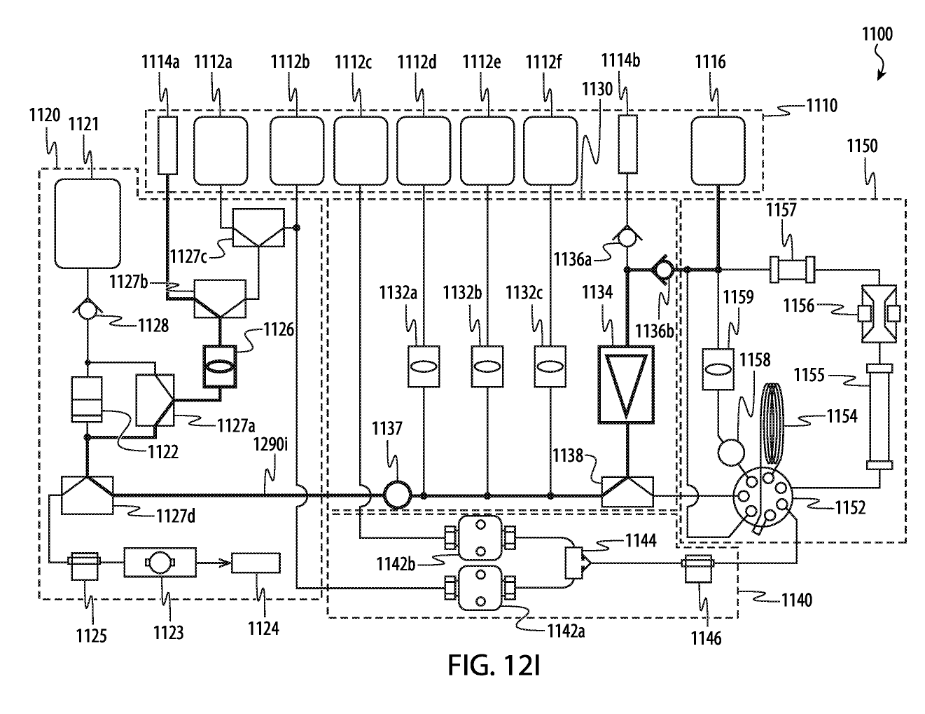

As described in greater detail below with respect to FIGS. 11-12I, the foregoing components and instruments may allow the sample capture module 320 to capture aldehydes from a breath sample, form an elution of the retained aldehydes, and clean or purge internal components of the sample capture module 320 or analysis device 300 more generally. For example, operation of the pump 424 may cause a breath sample held within the breath capture component 404 to progress through the directional valve 410, the cartridge 408 (and permeable membrane 412), multi-position valve 414, flow instrument 428, pump 424, and exhaust 416. This may allow the permeable membrane 412 to capture aldehydes from the breath sample. Subsequently, operation of the pump 454 may cause the first sample capture reagent 432 to progress through the multi-position valve 450, the multi-position valve 462, the pump 454, the multi-position valve 458, the cartridge 408 (and permeable membrane 412), the multi-position valve 414, and along the flow path F4 toward the mixing module 330. This may allow the sample capture module 320 to form an elution having the aldehydes of the breath sample that is delivered to the mixing module 330 for further processing into the mobile chromatography phase. Other modes or operations are contemplated and described herein, for example, such as using the pump 454 to dispense the second sample capture reagent 436 for cleaning or sanitizing of the sample capture module 320, among other appropriate functions.

FIG. 5 depicts sample components and instruments that may cooperate to implement and perform one or more functions of the mixing module 330. The various components and instruments described herein with respect to FIG. 5 are presented to facilitate an understanding of the mixing module 330 and are not meant as limiting. To the contrary, the described embodiment of FIG. 5 is intended to cover alternatives, modifications, and equivalents of the mixing module 330 consistent with the teachings herein.

The mixing module 330 may be configured to form a mobile chromatography phase that contains aldehydes from a breath sample. The mobile chromatography phase may be propagated through a stationary chromatography phase of the detection module 350, described herein, to detect an aldehyde content of the breath sample.

To facilitate the foregoing, the mixing module 330 may receive the flow F4 from the sample capture module 320. The flow F4 may include an elution having the aldehydes of the breath sample, as described above with respect to FIG. 4. Broadly, the elution may be advanced into a mixing volume 504 and mixed with one or more chemical compounds to form the mobile chromatography phase. For example, the mixing module 330 may be configured to advance air into the mixing volume 504 that agitates the elution and the one or more chemical compounds. Agitation from the air may mix the elution and the chemical compounds to form the mobile chromatography phase. The mobile chromatography phase may be advanced out of the mixing module 330 along a flow path F5 to the detection module 350.

To facilitate the foregoing, the mixing module 330 may be configured to selectively dispense reagents or other chemical compounds from the reagent module 310 that may facilitate in forming a mobile chromatography phase from the elution. As described above with respect to FIG. 3, the reagent module 310 may include multiple chemical compounds, filters, receptacles, and/or other compounds or structures. Shown in FIG. 5 are possible chemical compounds and filters that may be fluidically coupled with the mixing module 330. It will be appreciated, however, that the mixing module 330 may be fluidically coupled with more or fewer items of the reagent module 310. Further, reagent module 310 may include additional chemical compounds and structures, for example, such as those described herein with respect to FIGS. 4 and 6-8.

In the embodiment of FIG. 5, the mixing module 330 is fluidically coupled with a first mixing reagent 550, a second mixing reagent 554, a third mixing reagent 558, a first mixing filter 562, and a second mixing filter 566. The first mixing reagent 550 may be an internal standard or calibrant (having a known or predetermined aldehyde content), the second mixing reagent 554 may be a catalyst, the third mixing reagent 558 may be a dye (such as a fluorescent dye), and the first and second mixing filters 562, 566 may be air filters, such as a carbon-based filters; however, other chemical compounds and structures are possible. Broadly, the first mixing reagent 550 may include a known value of aldehydes. This known value may be used to establish a baseline or calibrated curve, by which other detected aldehydes of the breath sample may be compared. The second mixing reagent 554 may be used to initiate or accelerate a chemical reaction that forms the mobile chromatography phase from the elution. The third mixing reagent 558 may bond or attach to aldehydes within the elution. The dye may be responsive to radiation (e.g., such as increasing in brightness), thereby allowing for optical detection of aldehydes. The first mixing filter 562 may be used as an air intake for air agitation by the mixing volume 504. The second mixing filter 562 may be used as air intake for the mixing volume 504, for example, when the mobile chromatography phase is drawn from the mixing volume and along the flow path F5, among other functions.

One or more valves, pumps, and/or other components or instruments of the mixing module 330 may operate to selectively dispense the chemical compounds from the reagent module 310. As shown in FIG. 5, the mixing module 330 may include a first mixing pump 508. The first mixing pump 508 may be configured to dispense the first mixing reagent 550 into a flow that propagates into the mixing volume 504. The mixing module 330 may further include a second mixing pump 512. The second mixing pump 512 may be configured to dispense the second mixing reagent 554 into a flow that propagates into the mixing volume 504. The mixing module 330 may further include a third mixing pump 516. The third mixing pump 516 may be configured to dispense the third mixing reagent 558 into a flow that propagates into the mixing volume 504.

The first mixing pump 508, the second mixing pump 512, and the third mixing pump 516 may each be fixed volume (displacement) pumps configured to dispense a certain and controlled quantity of the respective mixing reagents into the elution. Other types of pumps may be used, including other fixed volume pumps, or pumps that may allow a predefined volume of fluid to pass for a given pump stroke or cycle. As shown in FIG. 5, the first mixing pump 508, the second mixing pump 512, and the third mixing pump 516 may introduce the respective mixing reagents into a flow of the elution as it propagates into the mixing volume 504. This may allow for premixing of the mixing reagents with the elution prior to mixing within the mixing volume 504 (e.g., using air agitation described below). In other cases, however, the mixing reagents may be introduced into the mixing volume 504 along a separate path from that of the elution. For example, mixing reagents and the elution may be introduced into the mixing volume 504 separately, including through distinct openings in the mixing volume 504, in other embodiments.

Each of the first mixing pump 508, the second mixing pump 512, and the third mixing pump 516 may dispense a corresponding one of the mixing reagents independent from one another. In this regard, the pumps may be tuned to control a flow rate of the mixing reagents into the elution, according to various parameters of the mobile chromatography phase. In one embodiment, the first mixing pump 508 may be calibrated to dispense 45 microliters of the first mixing reagent 550 into the elution as it flows toward the mixing volume 504. The second mixing pump 512 may be calibrated to dispense 45 microliters of the second mixing reagent 554 into the elution as it flows toward the mixing volume 504. The third mixing pump 516 may be calibrated to dispense 150 microliters of the third mixing reagent 558 into the elution as it flows toward the mixing volume 504. In other cases, more or less of the first mixing reagent 550, the second mixing reagent 554, and the third mixing reagent 558 may be added to the elution as may be appropriate to form the mobile chromatography phase.

The mixing module 330 may also include a fourth mixing pump 520. The fourth mixing pump 520 may be used to advance air into the mixing volume 504 for air agitation. For example, the fourth mixing pump 520 may be configured to draw air from atmosphere (and through the fluidically coupled first filter 562 of the reagent module 310) and direct the air toward the mixing volume 504. Upon entry, and as described in greater detail below with respect to FIG. 6, the air may bubble, percolate, or otherwise flow through the liquid solution held within the mixing volume 504. This may agitate or mix the liquids to facilitate formation of the mobile chromatography phase within the mixing volume 504.

As described in greater detail below with respect to FIGS. 11-12I, the foregoing components and instruments may facilitate operation of the mixing module 330 to receive an elution and mixing reagents, mixing the elution and mixing reagents, and flow a mobile chromatography phase formed from the elution and the reagents toward the detection module 350. For example, a gas sensor 524 (bubble detector) may detect introduction of the elution into the mixing module 330 along the flow path F4. This may initiate mixing reagent flow from one or more of the first mixing pump 508, the second mixing pump 512, and/or the third mixing pump 516, thereby allowing the first mixing reagent 550, the second mixing reagent 554, the third mixing reagent 558, and the elution to flow into the mixing volume 504. Subsequent to (or at least partially concurrent with) filling the mixing volume 504, the fourth mixing pump 520 may initiate a flow of air in the mixing volume 504, thereby causing air agitation within the mixing volume 504 that is used to form the mobile chromatography phase. The mixing volume 504 may be fluidically coupled with a multi-position valve 528. This may allow the mobile chromatography phase to exit the mixing volume 504 and flow along the flow path F5, for example, toward the detection module 350. The mixing volume 504 may also be fluidically coupled with the second filter 566. This may allow air from the atmosphere to be drawn into the mixing volume 504 as the mobile chromatography phase exits the mixing volume 504 and proceeds along the flow path F5.

FIG. 6 depicts another embodiment of the mixing module 330 described above with respect to FIGS. 3 and 5. In particular, FIG. 6 shows the mixing module 330 having a mixing volume 604. The mixing volume 604 may be substantially analogous to the mixing volume 504 described above with respect to FIG. 5. For example, the mixing module 604 may be configured to receive multiple mixing reagents and an elution (containing aldehydes) and form a mobile chromatography phase.

To facilitate the foregoing, the mixing volume 604 may be defined by angled sidewalls 674. The angled sidewalls 674 may extend away from a mixing opening 675 positioned at the bottom of the mixing volume 604. Accordingly, the angled sidewalls 674 may define a cone or contoured shape that expands outward from a bottommost portion of the mixing volume 604.

The angled sidewalls 674 that define the cone may facilitate mixing or air agitation. For example, FIG. 6 shows the mixing volume 604 in a configuration in which a solution 680 is contained therein. The solution 680 may be some combination of mixing reagents and elution that are combined to form the mobile chromatography phase. Air 682 (or other gas) may be introduced into the mixing volume 604 (from a flow path F6) through the mixing opening 675 and allowed to flow or percolate through the solution 680. In some cases, at least a portion of the air 682 may flow along the angled sidewalls 674, which may facilitate air agitation of the solution 680.

FIG. 6 also depicts the mixing volume 604 fluidically coupled with one or more structures of the reagent module 310. In the embodiment of FIG. 6, the reagent module 310 is shown as having a mixing filter 660 and a receptacle 670. However, as described above, the reagent module 310 may include multiple chemical compounds and structures. As such, structures in the reagent module 310 depicted in FIG. 6 are shown to illustrate one or more functions of mixing volume 604 and are not meant as limiting.

The mixing filter 660 may be an air filter fluidically coupled with the mixing volume 604 using a direction valve 662. The mixing filter 660 may be used to filter atmospheric air that may be drawn into the mixing volume 604 during one or more operations of the mixing module 330 (e.g., evacuating the mixing volume 604). The direction valve 662 may be a check valve or regulator that substantially prevents backflow or flow into the filter 660 from the mixing volume 604. The receptacle 670 may be a container, bin, vessel, or the like that captures an output of the mixing module 330, or the analysis device 300 more generally. The receptacle 670 may be fluidically coupled with the mixing volume 604 using a directional valve 672. The directional valve 672 may be a check valve or regulator that substantially prevents backflow or flow into the mixing volume 604 from the receptacle.

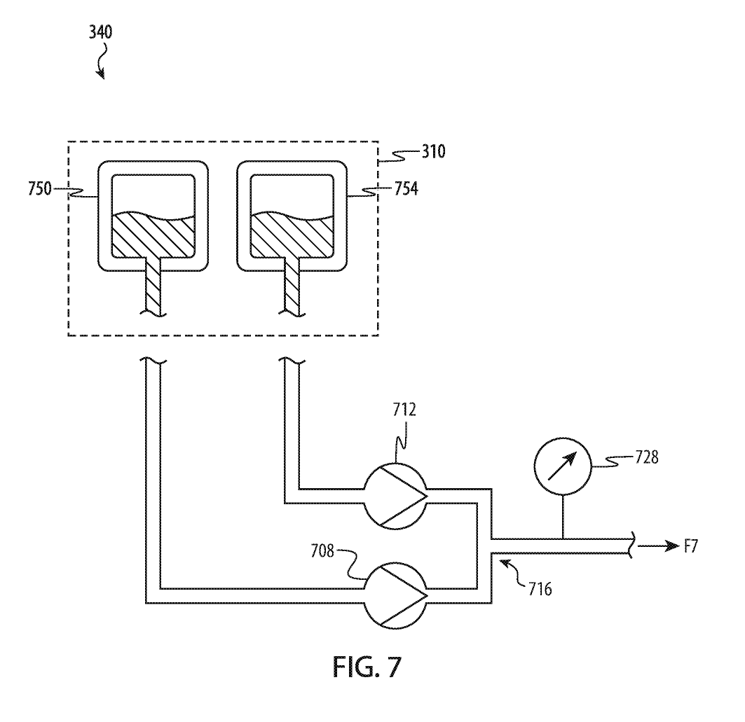

FIG. 7 depicts sample components and instruments that may cooperate to implement and perform one or more functions of the injection module 340. The various components and instruments described herein with respect to FIG. 7 are presented to facilitate an understanding of the injection module 340 and are not meant as limiting. To the contrary, the described embodiment of FIG. 7 is intended to cover alternatives, modifications, and equivalents of the injection module 340 consistent with the teachings herein.

The injection module 340 may be configured to form a pressurized combination of an injection reagent and a buffer. The pressurized combination may be output to the detection module 350 along a flow path F7. A concentration of the injection reagent may be tunable based on a flow rate of one or both of the injection reagent and the buffer as pumped along a direction toward the flow path F7. This concentration of the injection reagent may at least partially control the separation of the various distinct aldehydes groups of designations (e.g., C4, C5, C6, and so on), as described herein.

To facilitate the foregoing, the injection module 340 may be configured to selectively dispense reagents from the reagent module 310 that may facilitate forming a pressurized combination for use with the detection module 350. As described above with respect to FIG. 3, the reagent module 310 may include multiple chemical compounds, filters, receptacles, and/or other compounds or structures. Shown in FIG. 7 are possible chemical compounds and filters that may be fluidically coupled with the injection module 340. It will be appreciated, however, the mixing module 330 may be fluidically coupled with more or fewer items of the reagent module 310. Further, reagent module 310 may include additional chemical compounds and structures, for example, such as those described herein with respect to FIGS. 4-6 and 8.

In the embodiment of FIG. 7, the injection module 340 is fluidically coupled with a first injection reagent 750, and a second injection reagent 754. The first injection reagent 750 may be a reagent, such as MeOH 100% and the second injection reagent 754 may be a buffer. The concentration of the first injection reagent 750 may be reduced or otherwise controlled by the second injection reagent 754, as described herein (e.g., such as reducing MeOH concentration from 100% to 40%, or lower, based on the buffer).

One or more valves, pumps, and/or other components or instruments of the injection module 340 may operate to selectively dispense the chemical compounds from the reagent module 310. As shown in FIG. 7, the injection module 340 may include a first injection pump 708. The first injection pump 708 may be configured to dispense the first injection reagent 750 into a flow that propagates along the flow path F7. The injection module 340 may further include a second injection pump 712. The second injection pump 712 may be configured to dispense the second injection reagent 754 into a flow that propagates along the flow path F7. Both of the first injection pump 708 and the second injection pump 712 may be substantially high pressure pumps configured to increase pressure within (pressurize) and input flow. In one example, both of the first injection pump 708 and the second injection pump 712 may output flow at 2,000 pounds-per-square inch (psi). In other cases, the output may be more or less than 2,000 psi (including as high as 10,000 psi, or higher) and which may be variable based on a configuration of the analysis device 300. Further, it will be appreciated that the first injection pump 708 and the second injection pump 712 may operate to produce output having different pressures and/or different flow rates, as may be appropriate to produce the gradient ramp, described herein.

The first injection reagent 750 and the second injection reagent 754 may combine at a static mixing tee 716. The static mixing tee 716 may include an interior feature that facilitates blending of the first injection reagent 750 and the second injection reagent 754 (e.g., including an internal protrusion); however, this is not required. The first injection reagent 750 and the second injection reagent 754 may exit the static mixing tee 716 as a substantially combined flow that forms the pressurized combination output along the flow path F7.

A flow instrument 728 (including an in-line flow meter, or other gauge or instrument) may detect a flow rate of the pressurized combination output from the static mixing tee 716, or more generally, from the first injection pump 708 and the second injection pump 712. An output or signal of the flow instrument 728 may be transmitted to a processing unit of the analysis device 300 (or of another electronic device). This signal may be used to control or regulate one or more functions of the first injection pump 708 and the second injection pump 712, such as adjusting a flow rate and/or pressurized output in order to maintain a desired output along the flow path F7. The flow instrument 728 may also be used to detect a depressurization event of the injection module 340. This may occur, for example, when one or both of the first injection pump 708 or the second injection pump 712 cavitates or otherwise interacts with gasses (e.g., such as those trapped within the injection reagents. Such gasses may mitigate or prevent the pumps from maintaining adequate pressure along the flow path F7. Upon detection of depressurization, and as explained in greater detail below with respect to FIGS. 8 and 9, the analysis device 300 may transition into a configuration that primes or otherwise allows for fluid flow through the pumps.

As described herein, the first injection pump 708 and the second injection pump 712 may cooperate to form a pressurized combination having a concentration of reagent along a gradient ramp. For example, one or both of the flow rate (or other characteristic) of the first injection pump 708 and the second injection pump 712 may be modified in order to gradually increase a concentration of the reagent in the pressurized combination over time. It will be appreciated that while the first injection reagent 750 and the second injection reagent 754 are shown in FIG. 7 as being mixed or combined after pumping, in other cases the first injection reagent 750 and the second injection reagent 754 may be combined with one another before pumping. For such embodiment, a single pump may be used to pressurize the combination of the first injection reagent 750 and the second injection reagent 754 for transfer to the detection module 350.

FIG. 8 depicts sample components and instruments that may cooperate to implement and perform one or more functions of the detection module 350. The various components and instruments described herein with respect to FIG. 8 are presented to facilitate an understanding of the detection module 350 and are not meant as limiting. To the contrary, the described embodiment of FIG. 8 is intended to cover alternatives, modifications, and equivalents of the detection module 350 consistent with the teaching herein.

The detection module 350 may be configured to detect an aldehyde content of a patient breath sample. In particular, the detection module 350 may be configured to propagate a mobile (liquid) chromatography phase (containing aldehydes from the breath sample) through a stationary chromatography phase (high-density silica) in order to separate the aldehydes by molecule or group size or designation (e.g., aldehyde C4, C5, C6, and so on). The detection module 350 may also be configured to detect a value for each of the separated aldehydes using an excitation source (laser) to fluoresce dye attached to the aldehydes.

To facilitate the foregoing, the detection module 350 may include a control valve 804. Broadly, the control valve 804 may be configured to direct a mobile chromatography phase (e.g., from the mixing module 330) into a flow of a pressurized combination (e.g., from the injection module 340) that may operate to advance the mobile chromatography phase through the stationary chromatography phase. The control valve 804, as described in greater detail below with respect to FIG. 9A-9C, may include multiple ports (such as seven ports shown in FIG. 8). Particular ones of the multiple ports may be fluidically coupled to one another based on a configuration or position of the control valve 804. This may allow for operation of the control valve 804 in a first configuration to receive a volume of the mobile chromatography phase. In a second configuration, the control valve 804 may allow the volume of the mobile chromatography phase to be directed into a flow path of a pressured combination of flow that advances the mobile chromatography phase toward a column or other structure of an HPLC process. And in a third configuration, the control valve 804 may allow one or more fluid flows (e.g., such as the pressurized combination) to flow to waste, for example, as may be used to facilitate priming cavitated pumps of the injection module 340. It will be appreciated that while the control valve 804 described herein may be a seven-port, three-configuration valve, other valves may be used to implement the functions of the detection module 350 described herein, including a six-port, two-configuration valve, or other appropriate valve that may route an output of the mixing module to an output of the injection module.

In the embodiment of FIG. 8, the control valve 804 may receive the mobile chromatography phase from the mixing module 330 along the flow path F5 at a port 1. The control valve 804 may be coupled with a sample loop 808 that is fluidically coupled with a port 3 and port 6 of the control valve 804. The sample loop 808 may have an internal volume that is tunable to a desired volume of the mobile chromatography phase for separation by the HPLC process. For example, the sample loop 808 may have a volume of 800 microliters; however, in other cases the sample loop 808 may have a volume of more or less than 800 microliters. In a first configuration of the detection module 350, the sample loop 808 may be filled with the mobile chromatography phase. To facilitate the foregoing, the detection module 350 may include a detection pump 812. The detection pump 812 may be fluidically coupled to the control valve 804 at a port 2. The detection pump 812 may thus cause the mobile chromatography phase to flow from the port 1 to the port 3, fill the sample loop 808, and from the port 6 to the port 2. Excess volume of the mobile chromatography phase may flow, in one embodiment, to the reagent module 310, such as to receptacle 890. The receptacle 890 may be fluidically coupled to an output of the detection pump 812 and may be substantially analogous to the receptacle 670 described above with respect to FIG. 6. Fluidically coupled with the detection pump 812 may also be a gas sensor 816 (bubble detector). The gas sensor 816 may be used to detect the presence of gas or liquid at an inlet (or outlet) of the detection pump 812. In this regard, the gas sensor 816 may be used to detect a filling status of the sample loop 808. For example, when the gas sensor 816 no longer detects gas, or detects a minimal amount, or drop in gas content, the sample loop 808 may be substantially filled with the mobile chromatography phase. As such, the gas sensor 816 may be used to initiate a change in configuration of the control valve 804 (e.g., to the second configuration of the detection module 350) where the mobile chromatography phase within the sample loop 808 is directed toward a separation column and into a flow of the pressured combination.

The control valve 804 may receive a pressurized combination of reagent and buffer at a port 5, such as along the flow path F7 from the injection module 340. In a second configuration of the detection module 350, the port 5 may be fluidically coupled with the port 6 and the port 3 may be fluidically coupled with the port 4. As such, the pressurized combination received by the control valve 804 at the port 5 may advance the mobile chromatography phase out of the sample loop and toward a column 820. The column 820 may define a stationary chromatography phase of the HPLC process. For example, the column 820 may include one or separation substrates 824 (such as one or more layers of high-density silica) or other appropriate material. The separation substrates 824 may be permeable structures that impede advancement of aldehydes through the column 820. For example, and as described herein with respect to FIG. 3, the separation substrates 824 may permit advancement of aldehydes having a certain group or designation (e.g., aldehyde C4, C5, C6, and so on) at least partially based on a flow pressure at an inlet of the column 820 and a concentration of reagent (e.g., the pressurized combination received at the port 5). Both the low pressure and the reagent concentration may be controlled by the injection module 340 described above with respect to FIG. 7, and thus the injection module 340 may be used to selectively advance certain aldehydes through the column at specified time intervals. As such, molecules of a given aldehyde group may be advanced through, and output from the column, clustered with molecules of other like aldehyde groups. The cluster of molecules of like aldehyde groups may be separated from other, distinct aldehyde groups by a time interval as controlled by the injection module 340. Accordingly, aldehyde content may be measured at an output of the column and associated with a relative quantity of a particular aldehyde group.

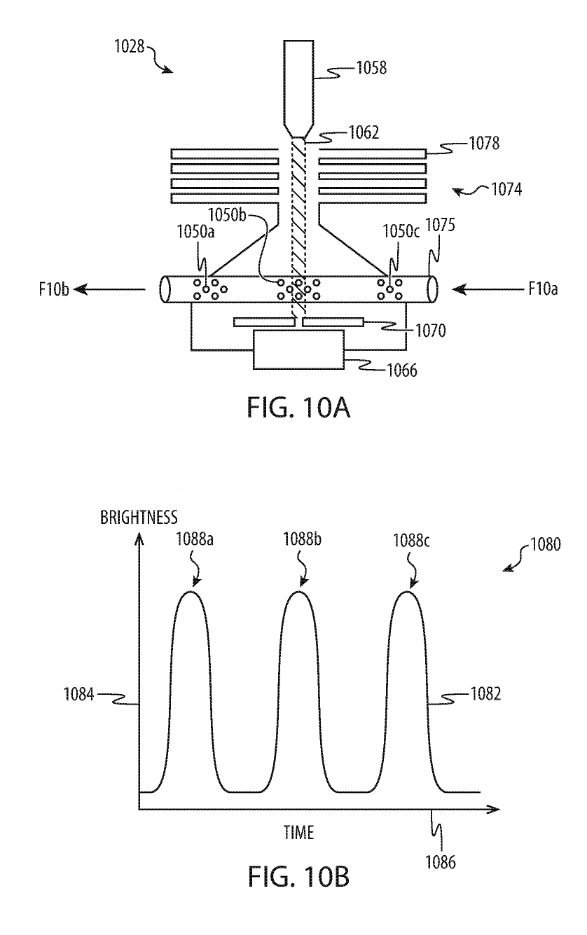

To facilitate the foregoing, the detection module 350 may include a detection assembly 828. The detection assembly 828, as described in greater detail below with respect to FIGS. 10A and 10B, may be configured to detect aldehydes at an output of the column 820. Broadly, the detection assembly 828 may include an excitation source (laser) and an optical detector, not shown in FIG. 8. The excitation source may expose the output of the column 820 to radiation, thus causing the dye attached to the aldehyde groups to fluoresce. This fluorescence may be detected by the detection assembly 828 and used to determine a relative value of a given aldehyde at the output of the column 820. The optical detector may generally measure a brightness of one or more aldehydes or aldehyde groups, although other detectors may measure other physical or chemical characteristics.

The detection module 350 may also include a regulator 832. The regulator 832 may be fluidically coupled to an output of the detection assembly 828. The regulator 832 may be a pressure regulator that is configured to maintain a minimum pressure at the output of the detection assembly 828. In this regard, the regulator 832 may be a pressure regulator that allows flow therethrough when a threshold pressure is satisfied. The regulator 832 may thus prevent the output of the detection assembly 828 from venting directly to atmospheric pressure, which may help reduce gas formation with the detection assembly 828.

As shown in FIG. 8, the control valve 804 also includes a port 7. In a third configuration of the detection module 350, the port 5 may be fluidically coupled with the port 7. In this regard, in the third configuration, the control valve 804 may direct the pressurized combination of the flow F7 to the port 7 and toward the reagent module 310 (such as toward the receptacle 890). This third configuration may be used to prime the injection pumps described above with respect to FIG. 7. For example, upon a detection of a depressurization of the pressured combination output by the injection module 340, the control valve 804 may be operated in the third configuration. This may reduce static pressure at the output of the pumps (e.g., by venting the pumps to atmospheric or near atmospheric pressure), and thereby help prime the pumps with the appropriate reagent and pressurize the output. Once primed, the control valve 804 may return to one of the first configuration or the second configuration of the detection module 350, as may be appropriate for a given application.

FIGS. 9A-9C depict various configurations of a control valve 904. The control valve 904 may be substantially analogous to the control valve 804 described above with respect to FIG. 8. For example, the control valve 904 may be a multi-position, multi-port valve that is used to direct a mobile chromatography phase (e.g., from the mixing module 330) into a flow of a pressurized combination (e.g., from the injection module 340) that may operate to advance the mobile chromatography phase through a stationary chromatography phase.

To facilitate the foregoing, the control valve 904, as shown in FIGS. 9A-9C, may have seven ports. Particular ones of the seven ports may be fluidically coupled with one another based on a configuration or position of the control valve 904. In one embodiment, the control valve 904 may alternate or transition between the respective configurations or positions in order to, for example, redirect or transfer a defined volume of fluid from a first flow or process (e.g., a relatively low pressure flow) into a second flow or process (e.g., a relatively high pressure flow). For example, FIGS. 9A-9C show a sample loop 908 having a defined volume that may be fluidically coupled with the control valve 904 in order to facilitate redirection or transfer of fluid between a relatively low pressure flow and a relatively high pressure flow. To facilitate the foregoing, the control valve 904 may be operated to allow the sample loop 908 to be filled in a first configuration using an output from a relatively low pressure flow and subsequently transferred in a second configuration to a relatively high pressure flow. The control valve 904 may further be operated in other configurations, including bypassing the sample loop 908, as described herein.

With reference to FIG. 9A, a control valve 904 is shown. In the first configuration 900a, the control valve 904 may be operated to load or fill the sample loop 908. The sample loop 908 may be filled using a flow F9a received by the control valve 904 at a first port. The flow F9a may include a chemical compound, solution, mobile chromatography phase, and so on, that is directed into the sample loop 908 when the control valve 904 is in the first configuration 900a. For example, when the control valve 904 is in the first configuration 900a, the first port may be fluidically coupled with a sixth port of the control valve 904. The sample loop 908 may be fluidically coupled to the sixth port and a third port of the control valve 904. Accordingly, when the control valve 904 is in the first configuration 900a, the flow F9a received at the first port may flow to the sixth port and fill the sample loop 908. The third port may be fluidically coupled with a second port of the control valve 904. Thus, the control valve 904 may output a flow F9b at the second port based on the sample loop 908 filling from the flow F9a received at the first port of the control valve 904.

In the embodiment of FIG. 9A, the flow F9a may generally correspond to an output from a mixing module, such as the mixing module 330 described herein with respect to FIGS. 3-8. The output of the mixing module 330 may be a mobile chromatography phase that contains aldehydes of a patient breath sample. The output may be a substantially low pressure output. The flow F9b may be fluidically coupled with a pump, such as the detection pump 812, described herein with respect to FIG. 8. Accordingly, suction or biasing from the pump (along the flow path F9b) may cause or initiate flow of the mobile chromatography phase along F9a and through the first port, the sixth port, the third port, and the second port, thereby filling the sample loop 908 with the output of the mixing module 330. One or more sensors (not shown in FIG. 9A) may detect or monitor filling of the sample loop 908. This may be used to trigger a subsequent configuration of the control valve 904.

With reference to FIG. 9B, a second configuration 900b of the control valve 904 is shown. In the second configuration 900b, the control valve 904 may be operated to insert or redirect sample loop 908 into another flow, such as a high pressure flow. For example, as shown in FIG. 9B, the control valve 904 may receive a flow F9c at a fifth port. The flow F9c may be a high pressure or other flow that is distinct from the flow F9a received by the control valve 904 at the first port (e.g., such as the pressurized combination output by the injection module 340 of FIG. 7). When the control valve 904 is in the second configuration 900b, the fifth port may be fluidically coupled with the sixth port. As described above, the sample loop 908 may be fluidically coupled with the sixth port and the third port of the control valve 904. Accordingly, when the control valve 904 is in the second configuration 900b, the flow F9c received at the fifth port may flow to the sixth port and through the sample loop 908. The third port may be fluidically coupled with a fourth port of the control valve 904. Thus the control valve 904 may output a flow F9d at the fourth port based on the sample loop 908 filling from the flow F9c received at the fifth port of the control valve 904.

As described above with respect to FIG. 9A, the sample loop 908 may be filled with a chemical compound or other substance received at the first port of the control valve 904 (e.g., a mobile chromatography phase). Thus, the control valve 904 may introduce the flow F9c into the substance contained within the sample loop 908 in the second configuration 900b. This may cause the contents of the sample loop 908 to exit the control valve 904 along the flow path F9d.

In the embodiment of FIG. 9B, the flow F9c may generally correspond to an output of an injection module, such as the injection module 340 described herein with respect to FIGS. 3-8. The output of the injection module 340 may be a pressurized combination of reagent and buffer. Correspondingly, the exit flow F9d may generally correspond to an inlet of a column or separation structure, such as the column 820 described with respect to FIG. 8, that may be used as a stationary chromatography phase of an HPLC process. The second configuration 900b may therefore be used to advance a substance or compound held by the sample loop 908 toward an inlet of a column. In this regard, where the sample loop 908 includes the mobile chromatography phase, the second configuration 900b may cause the pressurized combination (of the injection module 340) to flow from the fifth port, to the sixth port, through the sample loop 908 to the third port, to the fourth port, thereby causing the mobile chromatography solution to exit the control valve 904 at the flow F9d and toward the column.

With reference to FIG. 9C, a third configuration 900c of the control valve is shown. In the third configuration 900c, the control valve 904 may be operated to direct the flow F9c to a waste receptacle or otherwise vent to atmospheric or near atmospheric pressure. For example, in the third configuration 900c, the fifth port may be fluidically coupled to a seventh port of the control valve. A flow F9e may exit the control valve 904 at the seventh port, which, in certain embodiments, may function as a vent or relief of the flow F9c. The third configuration 900c also allows the flow F9c to bypass or otherwise flow through the control valve 904 without traversing the sample loop.

In the embodiment of FIG. 9C, the flow F9e may be coupled with a receptacle, such as receptacle 890 described with respect to FIG. 8, or other component that may serve as a vent or collection structure for waste fluids. As described with respect to FIG. 8, the third configuration 900c may allow the analysis device 300 to repressurize an output (pressurized combination of reagent and buffer) of the injection module 340. For example, in the event that one or more pumps of the injection module 340 cavitates or depressurizes, the third configuration 900c of the control valve may be triggered. By venting an output of the pumps to atmospheric or near atmospheric pressure, static pressure at the output of the pumps may be reduced, thereby helping to prime the pumps or otherwise pressurize the output.

FIG. 10A depicts a detection assembly 1028. The detection assembly 1028 may be substantially analogous to the detection assembly 828 described above with respect to FIG. 8. For example, the detection assembly 1028 may be configured to detect aldehydes output from a column (e.g., column 820 of FIG. 8). In particular, the detection assembly 1028 may detect an increase in brightness of particles that flow between an emitter (laser) and a detector. The emitter may radiate or impart energy to the particles that causes fluorescent ones of the particles (e.g., a fluorescent dye) to fluoresce (e.g., increase in brightness). Aldehydes may be attached or bonded to a fluorescent dye, and thus the detected increase in brightness may be associated with the presence of aldehydes. In some cases, the detection assembly 1028 may measure a degree or intensity of the increase in brightness of aldehydes (or aldehyde groups). This may be compared with a baseline brightness value (e.g., of a sample having a known aldehyde content) to determine the relative quantity or concentration of a given aldehyde sample.

To facilitate the foregoing, the detection assembly 1028 may include an emitter 1058. The emitter 1058 may be a laser, light, or other excitation source configured to emit energy toward a flow of particles. For example, the emitter 1058 is shown in FIG. 10A as emitting an output 1062. The output 1062 may generally correspond to the path of a laser or radiation source that is projected from the emitter 1058. The output 1062 may be detected by a detector 1066. The detector 1066 may be positioned along an opposing side of the particle flow of the emitter 1058 such that the particle flow passes between the emitter 1058 and the detector 1066 and through the output 1062.

The detector 1066 may be an optical sensor that measures changes in light; however, this is not required. In other embodiments, the detector 1066 may be another sensor responsive to aldehydes, configured to measure or detect various physical and/or chemical properties of aldehydes, or otherwise configured to measure changes in concentrations of aldehyde groups within a flow path. In the embodiment of FIG. 10A, changes in light measured by the detector 1066 may be associated with fluorescing of a dye attached to aldehydes in a flow of particles between the detector 1066 and the emitter 1058. In one embodiment, the detector 1066 may receive the output 1062 from the emitter 1058 and generate a signal in response to an interruption in the output 1062, such as an increase in brightness caused by the fluorescence of the dye. In other cases, the detector 1066 may be coupled with, or include, a filter 1070, such as a band-pass or other filter. The filter 1070 may be tuned in order to expose the detector 1066 to a set band of wavelengths (e.g., such as those corresponding to an increase in brightness), but otherwise block light or other radiation from the emitter 1058. This may allow the detector 1066 to detect light produced by the fluorescence of particles, but otherwise be blocked by the filter 1070 from detecting light or other radiation of output 1062 absent the fluoresced particles.

To illustrate, in one embodiment, the emitter 1058 may be a laser. The laser may be configured to produce the output 1062. The output 1062 may be a beam having a predefined wavelength, such as 520 nanometers; however, other wavelengths are possible. The filter 1070 may be configured to allow energy having a range of certain wavelengths to pass therethrough. For example, the filter 1070 may allow wavelengths of greater than 520 nm to 540 nm to pass therethrough. Other wavelengths may be substantially blocked. The range of certain wavelengths allowed to pass through the filter 1070 may generally correspond to a wavelength of energy emitted by fluoresced particles. Accordingly, when the detector 1066 detects light through the filter 1070, the light may be from the fluoresced particles, and therefore used to determine an associated aldehyde content of the flow, as described herein.

The detection assembly 1028 may include a housing 1074. The housing 1074 may generally be used to support the emitter 1058 and the detector 1066 within the detection assembly 1028 relative to a flow of particles. In one embodiment, particles may flow through the housing 1074 along or within a through portion 1075. The through portion 1075 may be a fully or partially transparent passage or conduit of the housing 1074 that extends along a path substantially between the emitter 1058 and the detector 1066. This may allow the emitter 1058 to direct the output 1062 toward the through portion 1075 in order to fluoresce particles contained therein. The detector 1066, positioned along the through portion 1075 opposite the emitter 1058, may register or detect the corresponding changes in brightness caused by the fluoresced particles.

In a sample embodiment, the through portion 1075 may be configured to receive a flow F10a. The flow F10a may be an output from a column (e.g., column 820 of FIG. 8) or other separation structure of an HPLC process that contains aldehydes of a patient breath sample. Accordingly, as described above with respect to FIG. 8, the flow F10a may include clusters of distinct aldehyde groups that are separated from one another. In the embodiment of FIG. 10A, the flow F10a may include a first aldehyde group cluster 1050a, a second aldehyde group cluster 1050b, and a third aldehyde group cluster 1050c, each of which may be directed by the through portion 1075 within the housing 1074 and expelled at a flow F10b. Each of the first aldehyde group cluster 1050a, the second aldehyde group cluster 1050b, and the third aldehyde group cluster 1050c may include a fluorescent dye. As such, when a given one of the aldehyde group clusters passes through the output 1062, the detector 1066 may detect a fluoresce of the dye.

The housing 1074 may also include various other structures that help facilitate the operation of the detection assembly 1028. For example, the housing 1074 may define a heat sink 1078. The heat sink 1078 may be fins or other structures configured to radiate heat away from the emitter 1058. This may help reduce excess heat in the detection assembly 1028, which may enhance the reliability and longevity of various components of the analysis device, including the detector 1066. Other structures may be defined by the housing 1074 too, such as those configured to receive and/or position the emitter 1058 relative to the detector 1066.

FIG. 10B depicts a brightness-time diagram 1080. The brightness-time diagram 1080 depicts a sample output from the detector 1066 or other detector of the detection assembly 1028 described with respect to FIG. 10A. In particular, the brightness-time diagram 1080 depicts a curve 1082 that represents a brightness of light detected by detector 1066. The brightness may correspond to fluoresced light, such as that emitted by a fluorescent dye when hit by an excitation source, or otherwise impacted from radiation.

The brightness-time diagram 1080 may include a brightness axis 1084 and a time axis 1086. The brightness axis 1084 may generally represent an amount of light detected by the detector 1066. The amount of light may correspond to an intensity of light (e.g., degree of brightness) detected by the detector 1066 as measured over a period of time, represented by the time axis 1086. As described above with respect to FIG. 10A, clusters of like aldehyde groups may flow proximate the detector 1066 (e.g., such as within a field of the detector 1066) and fluoresce when hit by the emitter 1058 with radiation. Accordingly, the curve 1082 may include multiple peaks that represent an increase in brightness caused by the fluorescence of the cluster of like aldehyde groups. As shown in the embodiment of FIG. 10B, the curve 1082 may include a first peak 1088a, a second peak 1088b, and a third peak 1088c, each of which may correspond to an increase in brightness or radiation measured by the detector 1066 from a distinct aldehyde cluster.

To illustrate, and with reference to FIG. 10A, the first peak 1088a may generally correspond to the first aldehyde group cluster 1050a, the second peak 1088b may generally correspond to the second aldehyde group cluster 1050b, and the third peak 1088c may generally correspond to the third aldehyde group cluster 1050c. In this regard, the first peak 1088a may represent an increase in brightness measured by the detector 1066 when the first aldehyde group cluster 1050a is impacted by the output 1062, the second peak 1088b may represent an increase in brightness measured by the detector 1066 when the second aldehyde group cluster 1050b is impacted by the output 1062, and the third peak 1088c may represent an increase in brightness measured by the detector 1066 when the third aldehyde group cluster 1050c is impacted by the output 1062.

Broadly, each of the respective peaks of the curve 1082 may be associated with a particular aldehyde group or designation (e.g., aldehyde C4, C5, C6, etc.) based on the occurrence of the peak for a given time (e.g., as represented by the time axis 1086). For example, as described above with respect to FIGS. 3 and 8, the aldehyde group cluster may pass through the column 820 of FIG. 8 at specified intervals based on various process factors, including the pressurized output of, for example, the injection module 340 of FIGS. 3-8. These process factors may be controlled or tuned in order to anticipate a time, order, sequence, and/or other distinguishable pattern of aldehyde group cluster propagated from the column 820.

As a non-limiting illustration, the process factors may be tuned such that aldehyde group clusters of increasing molecule size (e.g., C4, C5, C6) are emitted from the column 820 and separated from one another by an interval of 30 seconds. A processing unit of the analysis device 300 (or of another electronic device) may therefore associate each of the peaks of the curve 1082 with an aldehyde group cluster based on a processing time measured along the time axis 1086. This may allow the analysis device 300 to determine, continuing the non-limiting illustration, that the first peak 1088a corresponds to a C4 aldehyde, the second peak 1088b corresponds to a C5 aldehyde, the third peak 1088c corresponds to a C6 aldehyde, and so on, as one example. In other cases, the peaks of the curve 1082 may correspond to other aldehyde groups or designations based on the value of the respective peak relative to the time axis 1086.

An amplitude of a peak of the curve 1082 (e.g., the first peak 1088a, the second peak 1088b, the third peak 1088c) may be analyzed to determine a relative value (quantity, amount, concentration) of the associated aldehyde group cluster. For example, the curve 1082 may be integrated relative to each of the detected peaks to determine a value of each of the associated aldehyde group clusters. While the peaks of the curve 1082 are shown in FIG. 10B as having similar amplitude for the purposes of the illustration, it will be appreciated that a patient breath sample may have peaks of varying or distinct amplitudes, and thus each value of the associated aldehyde group cluster may be different from one another. These values may be compared with a baseline or calibrant in order to determine a relative aldehyde content of each of the detected aldehyde group clusters. For example, one or more of the aldehyde group clusters may be associated with, or derived from, a calibrant (e.g., as described above with respect to FIGS. 3-8). The calibrant may have a known or standardized aldehyde content of a certain aldehyde group, such as an aldehyde group that is distinct from that which may be found in a patient breath sample. This calibrant may be added to the mobile chromatography phase, separated through the HPLC process, and detected by the detector 1066. The amplitude of the peak associated with the aldehyde group cluster (from the calibrant) may serve as a reference point for determining a relative concentration or aldehyde content of the other detected aldehydes. This relative concentration may be output to a user (e.g., using graphical outputs) and transmitted to another electronic device to assist in diagnosing certain medical conditions. In some cases, the analysis device 300 may determine an aldehyde score or other composite or derived metric using some or all of the relative concentrations.

FIG. 11 depicts a sample piping and instrument diagram for an analysis device 1100. The analysis device 1100 may be substantially analogous to the analysis device 300 described above with respect to FIG. 3. For example, the analysis device 1100 may include a reagent module 1110, a breath capture module 1120, a mixing module 1130, an injection module 1140, and a detection module 1150. As described above with respect to FIG. 3, the various modules of the analysis device 1100 represent collections of mechanical components, instruments, and so on that collectively operate to perform the various functions described herein. Rather than define discrete or separated mechanical components or instruments, the modules may be fluidically coupled with one another and operate using various combinations of common, overlapping, and/or interconnecting components and instruments. FIG. 11 depicts one embodiment of components and instruments that may be used to fluidically couple and operate the reagent module 1110, the breath capture module 1120, the mixing module 1130, the injection module 1140, and the detection module 1150.

The reagent module 1110 may be substantially analogous to the reagent module 310 described above with respect to FIGS. 3-8. For example, the reagent module 1110 may be configured to include some or all of the chemical compounds, filters, receptacles, and so on used by the analysis device 1100 for detection of the aldehyde content of a breath sample. In this regard, analogous to the components described in relation to the embodiments of FIGS. 3-8, the reagent module 1110 may include a first reagent 1112a, a second reagent 1112b, a third reagent 1112c, a fourth reagent 1112d, a fifth reagent 1112e, a sixth reagent 1112f, a first filter 1114a, a second filter 1114b, and a receptacle 1116, among other components. Redundant explanation of this feature is omitted here for clarity.

The breath capture module 1120 may be substantially analogous to the sample capture module 320 described above with respect to FIGS. 3-8. For example, the breath capture module 1120 may be configured to capture aldehydes from a breath sample and elute the captured aldehydes. In this regard, analogous to the components described in relation to the embodiments of FIGS. 3-8, the breath capture module 1120 may include a breath capture component 1121, a cartridge 1122, a vacuum pump 1123, a pan 1124, a flow instrument 1125, a fixed volume pump 1126, a first multi-position valve 1127a, a second multi-position valve 1127b, a third multi-position valve 1127c, a fourth multi-position valve 1127d, and a directional valve 1128, among other components. Redundant explanation of this feature is omitted here for clarity.

The mixing module 1130 may be substantially analogous to the mixing module 330 described above with respect to FIGS. 3-8. For example, the mixing module 1130 may be configured to form a mobile chromatography phase using the elution formed by the breath capture module 1120. In this regard, analogous to the components described in relation to the embodiments of FIGS. 3-8, the mixing module 1130 may include a first mixing pump 1132a, a second mixing pump 1132b, a third mixing pump 1132c, a mixing volume 1134, a first directional valve 1136a, a second directional valve 1136b, a flow instrument 1137, and a multi-position valve 1138, among other components. Redundant explanation of this feature is omitted here for clarity.

The injection module 1140 may be substantially analogous to the injection module 340 described above with respect to FIGS. 3-8. For example, the injection module 1140 may be configured to form a pressurized flow that is used to advance the mobile chromatography phase through the detection module 1150. In this regard, analogous to the components described in relation to the embodiments of FIGS. 3-8, the injection module 1140 may include a first injection pump 1142a, a second injection pump 1142b, a mixing tee 1144, and a flow instrument 1146, among other components. Redundant explanation of this feature is omitted here for clarity.

The detection module 1150 may be substantially analogous to the detection module 350 described above with respect to FIGS. 3-8. For example, the detection module 1150 may be configured to detect an aldehyde content of a patient breath sample using an output of the injection module 1140 and the mixing module 1130. In this regard, analogous to the components described in relation to the embodiments of FIGS. 3-8, the detection module 1150 may include a control valve 1152, a sample loop 1154, a column 1155, a detection assembly 1156, a regulator 1157, a flow instrument 1158, and a detection pump 1159, among other components. Redundant explanation of this feature is omitted here for clarity.

FIGS. 12A-12I depict various operations of the analysis device 1100. In particular, FIGS. 12A-12I represent various fluid flows through one or more of the reagent module 1110, the breath capture module 1120, the mixing module 1130, the injection module 1140, and the detection module 1150. Each of the fluid flows may correspond to a configuration of the analysis device 1100 that is one of a multi-step, integrated process that captures aldehydes from a patient breath sample and determines an aldehyde content using an unattended HPLC process. It will be appreciated that the flows described with respect to FIGS. 12A-12I are presented for purposes of illustration only. Other flows and configurations are contemplated.

With reference to FIG. 12A, a flow 1290a of the analysis device 1100 is depicted. The flow 1290a may correspond to an operation of capturing aldehydes from a patient breath sample on a permeable membrane. Accordingly, as shown in FIG. 12A, the flow 1290a may represent a flow of a patient breath sample from the breath capture component 1121 to the pan 1124.

With reference to FIG. 12B, a flow 1290b of the analysis device 1100 is depicted. The flow 1290b may correspond to an operation of forming an elution from the captured aldehydes of the patient breath sample. Accordingly, as shown in FIG. 12B, the flow 1290b may represent a flow of reagent from the first reagent 1112a through the cartridge 1122 (eluting the aldehydes) and to the mixing volume 1134. The flow 1290b may also represent a flow of further reagents (e.g., fourth reagent 1112d, fifth reagent 1112e, sixth reagent 1112f), including catalysts, calibrants, dyes, and so on, into the mixing volume 1134.

With reference to FIG. 12C, a flow 1290c of the analysis device 1100 is depicted. The flow 1290c may correspond to an operation of mixing the chemical compounds contained with the mixing volume 1134 with air (air agitation), which may help form the mobile chromatography phase. Accordingly, as shown in FIG. 12C, the flow 1290c may represent a flow of air from the first filter 1114a to the mixing volume 1134.

With reference to FIG. 12D, a flow 1290d of the analysis device 1100 is depicted. The flow 1290d may correspond to an operation of forming a pressurized combination of reagent and buffer. Accordingly, as shown in FIG. 12D, the flow 1290d may represent a flow of the second reagent 1112b and the third reagent 1112c into the control valve 1152 using the first injection pump 1142a and the second injection pump 1142b, respectively.

With reference to FIG. 12E, a flow 1290e of the analysis device 1100 is depicted. The flow 1290e may correspond to an operation of loading a mobile chromatography phase into a fixed volume. Accordingly, as shown in FIG. 12E, the flow 1290e may represent a flow of the mobile chromatography phase into the sample loop 1154 from the mixing volume 1134. The flow 1290e may be initiated by the detection pump 1159 when the control valve 1152 is in a first configuration (e.g., configuration 900a described with respect to FIG. 9A).

With reference to FIG. 12F, a flow 1290f of the analysis device 1100 is depicted. The flow 1290f may correspond to an operation of pushing the mobile chromatography phase through a column or other separation structure of the detection module 1150. Accordingly, as shown in FIG. 12F, the flow 1290f may represent a flow of the pressurized combination output from the injection module 1140 into the sample loop 1154, through the column 1155, detection assembly 1156, and to the receptacle 1116 of the reagent module 1110. The flow 1290f may be initiated when the control valve 1152 is in a second configuration (e.g., configuration 900b described with respect to FIG. 9B).

With reference to FIG. 12G, a flow 1290g of the analysis device 1100 is depicted. The flow 1290g may correspond to an operation of priming or repressurizing an output of the injection module 1140. Accordingly, as shown in FIG. 12G, the flow 1290g may represent a flow of an output of the injection module 1140 through the control valve 1152 and to the receptacle 1116. The flow 1290g may be initiated when the control valve 1152 is in a third configuration (e.g., configuration 900b described with respect to FIG. 9C).

With reference to FIG. 12H, a flow 1290h of the analysis device 1100 is depicted. The flow 1290h may correspond to an operation of sanitizing or otherwise flushing one or more components of the analysis device 1100 with a reagent. For example, this may be subsequent to an analysis of aldehydes in a first breath sample, in order to prepare the analysis for an analysis of aldehydes in a second or subsequent breath sample. Accordingly, as shown in FIG. 12H, the flow 1290h may represent a flow of the second reagent 1112b through the breath capture module 1120, the mixing module 1130, and to the receptacle 1116 of the reagent module 1110.

With reference to FIG. 12I, a flow 1290i of the analysis device 1100 is depicted. The flow 1290i may correspond to an operation of purging or otherwise passing air through one or more components of the analysis device 1100. For example, this may be used in order to prepare the analysis device 1100 for an analysis of aldehydes in a second or subsequent breath sample. Accordingly, as shown in FIG. 12I, the flow 1290i may represent a flow of air through the first filter 1114a and through the breath capture module 1120, the mixing module 1130, and to the receptacle 1116 of the reagent module 1110.

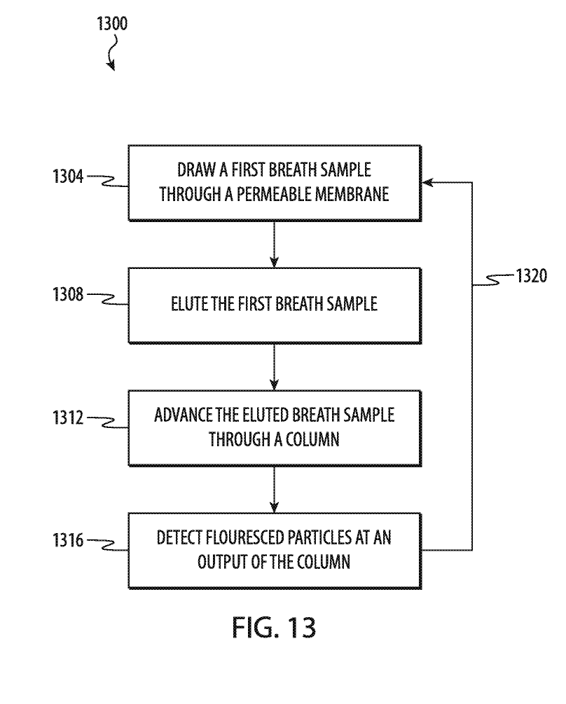

In this regard, with reference to FIG. 13, process 1300 relates generally to determining an aldehyde content of multiple breath samples. The process 1300 may be used with any of the breath analysis systems and breath analysis devices described herein, for example, such as breath analysis system 100, analysis device 104 and 300 and variations and embodiments thereof.

To facilitate the reader's understanding of the various functionalities of the embodiments discussed herein, reference is now made to the flow diagram in FIG. 13, which illustrates process 1300. While specific steps (and orders of steps) of the methods presented herein have been illustrated and will be discussed, other methods (including more, fewer, or different steps than those illustrated) consistent with the teachings presented herein are also envisioned and encompassed with the present disclosure.

At operation 1304, a first breath sample of multiple breath samples may be drawn through a permeable membrane of an analysis device. For example and with reference to FIG. 4, a breath sample contained within the breath capture component 404 may be drawn through the permeable membrane 412 of the analysis device 300. A pump 424, in one embodiment, may be a vacuum pump that is used to evacuate the breath sample from the breath capture component 404 and through the permeable membrane 412. The permeable membrane 412 may be a silica bed or other structure that may capture aldehydes from the breath sample as it is drawn through.

At operation 1308, a breath sample may be eluted from the permeable membrane using a first reagent from a container positioned within the analysis device. For example and with reference to FIG. 4, the second sample capture reagent 436 may be propagated through the permeable membrane 412. The second sample capture reagent 436 may elute aldehydes captured by the permeable membrane 412 as it flows through the cartridge. The elution may be advanced toward a mixing module (e.g., mixing module 330 of FIG. 5) in order to form a mobile chromatography phase that contains the aldehydes from the patient breath sample.

At operation 1312, an eluted breath sample may be advanced through a column using a second reagent from a container within the analysis device. For example and with reference to FIG. 8, the eluted breath sample may be part of a mobile chromatography phase that is advanced through the column 820 by the pressurized combination output from the injection module 340. For example, the mobile chromatography phase (formed at least partially from the eluted breath sample) may be loaded into the sample loop 808 and advanced through the column 820 by the flow F7, which may be an output of the injection module 340.

At operation 1316, fluoresced particles may be detected at an output of the column corresponding to an aldehyde content of the breath sample. For example and with reference to FIG. 8, the output of the column 820 may be analyzed by a detection assembly 828. The detection assembly 828 may detect fluoresced light emitted by fluorescent dye when the dye receives energy from an emitter or other energy source (e.g., as described in great detail above with respect to FIGS. 10A and 10B).

At operation 1320, the operations 1304-1316 may be repeated for a second breath sample. For example and with reference to FIGS. 3-8, the analysis device 300 may be configured for multiple, successive analyses of patient breath samples. For example, the analysis device 300 may be used in a clinical setting in which multiple samples are collected from patients and analyzed using the analysis device 300 by substantially untrained personnel. To facilitate the foregoing, the analysis device 300 may be configured to reset or return to an initial configuration in order to analyze subsequent breath samples. In one embodiment, this may involve flushing an internal network of tubes with further reagents from the container of the analysis device 300, for example, which may help sanitize or sterilize the analysis device 300 so that the aldehydes of the previous sample do not influence the detection of aldehydes in a subsequent breath sample. Further, the internal network tubes may also be purged with air filtered through the container, such as through the filter 440. This may help dry various components of the analysis device 300, prior to receiving reagents for the analysis of aldehydes in a subsequent breath sample.

As described herein, the analysis device 300 may use various chemical compounds or reagents to determine an aldehyde content of a breath sample. The reagents may be contained within a container of the analysis device 300. For example, as described in greater detail below, the analysis device 300 may include a container having internal chambers that may hold the reagents used by the analysis device 300 for the determination of an aldehyde content of the breath sample. In the sample process 1300, the container may include a quantity of at least a first reagent and a second reagent for the first breath sample and the second breath sample. Accordingly, the analysis device 300 may operate to determine an aldehyde content of multiple patient breath samples using the same container. This may facilitate use of the analysis device for multiple, successive analyses, for example, by reducing an interval for maintaining or restocking the analysis device 300 with additional chemical compounds. For example, the container may include sufficient reagents so that the analysis device 300 may analyze breath samples of each patient of a clinician on a given day or week, as one possibility.

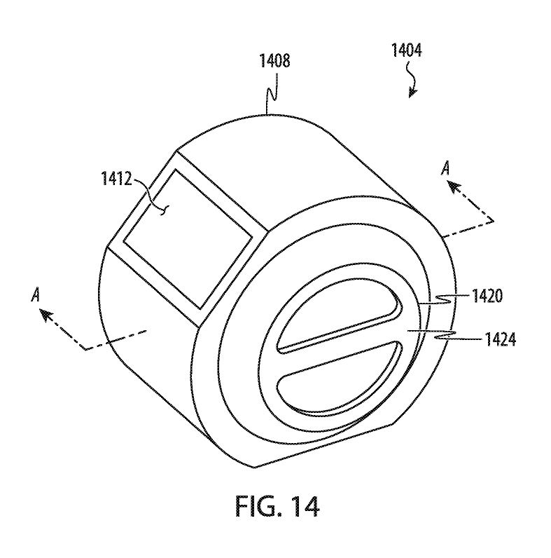

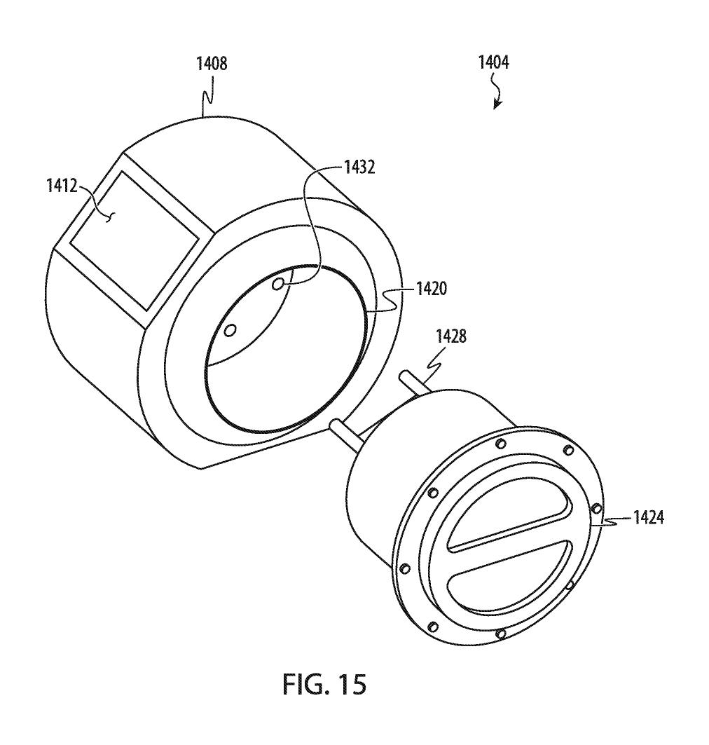

FIGS. 14-17 depict an analysis device 1404. The analysis device 1404 may be substantially analogous to the analysis device 104 described above with respect to FIGS. 1-2E. For example, the analysis device 1404 may be configured to determine an aldehyde content of a breath sample. Similar to the analysis device 104, the analysis device 1404 may include an unattended HPLC process and one or more systems that capture and elute aldehydes of a breath sample and detect aldehydes according to molecule size once separated by the HPLC. In this regard, analogous to the components described in relation to the embodiments of FIGS. 1-2E, the analysis device 1404 may include an enclosure 1408 and a display 1412, among other components. Generally, the enclosure 1408 may form an external surface of the analysis device 1404 that conceals various components, modules, and systems of the analysis device 1404. The display 1412 may be a touch sensitive display configured to depict an output of the analysis device 1404 corresponding to a detected aldehyde content. Redundant explanation of these features is omitted here for clarity.

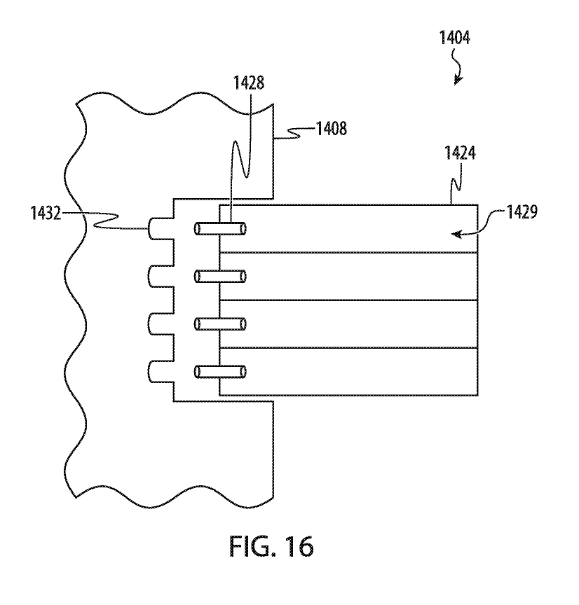

With reference to FIG. 14, the analysis device 1404 is shown having a container 1424 at least partially received within an opening 1420 of the enclosure 1408. The container 1424 may be a reagent container that is configured to hold reagents, chemical compounds, or other substances or solutions within internal chambers defined within an internal volume of the container 1424 (e.g., as described below with respect to FIG. 16). When in the assembled configuration shown in FIG. 14, the chemical compounds held within the container 1424 may be substantially concealed by the enclosure 1408 or otherwise positioned within the analysis device 1404.

With reference to FIG. 15, the analysis device 1404 is shown having the container 1424 removed from the opening 1420. As described herein, the container 1424 may be a removable component of the analysis device 1404. This may allow the container 1424 to include a quantity of reagents used for multiple breath analyses. When one or more of the reagents held within the container has a volume insufficient for subsequent breath analyses, the container 1424 may be replaced with a new container having a replenished quantity of reagents.

The container 1424 may be coupled to the analysis device 1404 using a variety of different structures and assemblies. For example, one or more fasteners, clips, guides, protrusions, and/or other attachment structures, and so on may be used to removeably couple the container to the analysis device 1404. In one embodiment, such attachment structures may be configured to secure the container 1424 within the opening 1420 upon rotating the container 1424 by a predetermined amount, such as a 45 degree quarter turn, when the container 1424 is at least partially received within the opening 1420. To disengage the container 1424 from the analysis device 1404, a user may rotate the container by a predetermined amount, such as a 45 degree quarter turn, in an opposing direction from the input used to attach the container 1424 within the analysis device 1404.

The analysis device 1404 may be configured to selectively dispense reagents or other chemical compounds from the container 1424 in order to perform one or more of the functions described herein. To facilitate the foregoing, the container 1424 may include a group of passages 1428. The group of passages 1428 may be configured to fluidically couple with a corresponding group of receiving features 1432 of the analysis device 1404. Each of the passages 1428, as described below with respect to FIG. 16, may be fluidically coupled with an internal chamber configured to hold a distinct reagent. Accordingly, when the container 1424 is advanced at least partially into the opening 1420, the group of receiving features 1432 may dispense particular reagents from the container 1424 using a corresponding one of the group of passages 1428. As described above, for example with respect to FIGS. 3-8, the analysis device 300 may selectively dispense reagents (e.g., using fixed volume pumps), and thus it will be appreciated that the group of receiving features 1432 shown in FIG. 15 may be fluidically coupled with appropriate components, instruments, devices, and so on, to facilitate such functionality.

With reference to FIG. 16, a simplified cross-sectional view of the analysis device 1404 and the container 1424 is shown. In particular, FIG. 16 illustrates the container 1424 having a group of internal chambers 1429. The group of internal chambers 1429 may be cavities or internal volumes of the container 1424 that are separated from one another. In this regard, each chamber of the group of internal chambers 1429 may be configured to hold a distinct chemical compound. A respective passage of the group of passages 1428 may be fluidically coupled to corresponding ones of the group of internal chambers 1429. Accordingly, when in an assembled configuration, a chemical compound held within an internal chamber of the container 1424 may be dispensed by the analysis device 1404 through the corresponding one of the group of passages 1428 and into an associated receiving feature.

It will be appreciated that the simplified cross-section of the container 1424 depicted in FIG. 16 is presented for purposes of illustration only. Internal chambers of the group of internal chambers 1429 may each have a distinct size, shape, and configuration, for example, based on a type of reagent held therein. In this regard, each of the internal chambers of the group of internal chambers 1429 may be configured for use with a certain chemical compound, and therefore may include or be coupled with a coating, material, and so on tailored for the chemical compound. Further, as described above with respect to FIGS. 3-8, the container 1424 (e.g., reagent module 310 of FIG. 3) may include other structures and devices, including filters, receptacles, and so on, not shown in FIG. 16 for the interest of clarity.

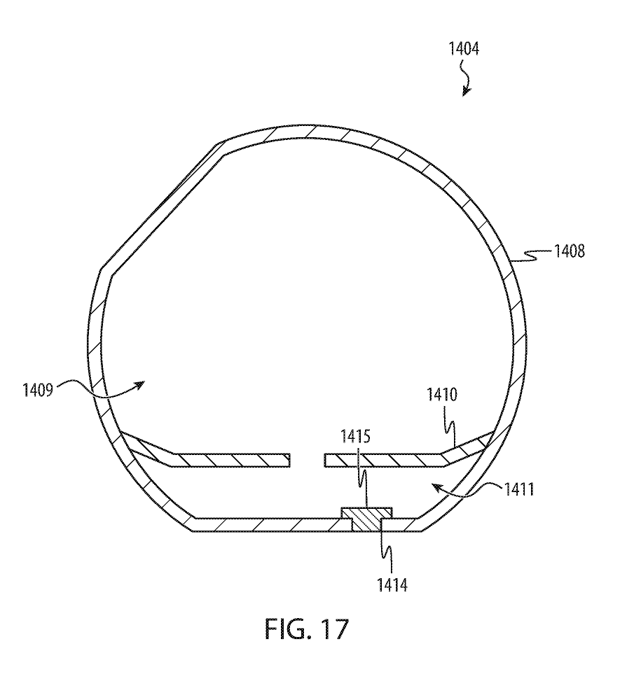

FIG. 17 depicts a cross-sectional view of the analysis device 1404 of FIG. 14, taken along line A-A of FIG. 14. As shown in FIG. 17, the enclosure 1408 may define an internal volume 1409 of the analysis device. While not shown in FIG. 17 for the interest of clarity, some or all of the components of, with reference to FIG. 3, the reagent module 310, the sample capture module 320, the mixing module 330, the injection module 340, and/or the detection module 350 may be positioned or concealed by the enclosure 1408.

The analysis device 1404 may include a false bottom 1410 positioned within the internal volume 1409 and coupled to the enclosure 1408. The false bottom 1410 may be used to channel stray fluids within the internal volume 1409 toward a collection volume 1411. Stray fluids may be caused, for example, in the event of leak or other failure of the various components of the reagent module 310, the sample capture module 320, the mixing module 330, the injection module 340, and/or the detection module 350, described herein. The internal volume 1409 may be substantially sealed from an external environment, and thus stray fluids may migrate toward the false bottom 1410 and into the collection volume 1411.

Stray fluids may thus pool or build up in the collection volume 1411. The collection volume 1411 may be substantially sealed from the external environment, which may help prevent or mitigate leaks from the analysis device 1404. Fluids held within the collection volume 1411 may be evacuated, for example, in order to perform maintenance on the analysis device 1404, transport the analysis device 1404, and so on. To facilitate the foregoing, the enclosure 1408 may define an outlet 1415. The outlet 1415 may fluidically couple the collection volume 1411 with the external environment. A plug 1414, or other feature configured to temporarily seal the collection volume 1411 may be positioned within the outlet 1415. Accordingly, a user may remove the plug 1414 from the outlet 1415 in order to empty fluids from the collections volume 1411.

FIG. 18 presents an illustrative analysis device 1800. The schematic representation in FIG. 18 may be substantially analogous to the analysis device 104 and 300 described above with respect to FIGS. 1 and 3. However, FIG. 18 may also more generally represent other types of devices and configurations that may be used to receive a user input signal from an input device in accordance with the embodiments described herein. In this regard, the analysis device 1800 may include any appropriate hardware (e.g., computing devices, data centers, switches), software (e.g., applications, system programs, engines), network components (e.g., communication paths, interfaces, routers) and the like (not necessarily shown in the interest of clarity) for use in facilitating any appropriate operations disclosed herein.

As shown in FIG. 18, the analysis device 1800 may include a processing unit or element 1808a operatively connected to computer memory 1812 and computer-readable media 1816. The processing unit 1808a may be operatively connected to the memory 1812 and computer-readable media 1816 components via an electronic bus or bridge (e.g., such as system bus 1810). The processing unit 1808a may include one or more computer processors or microcontrollers that are configured to perform operations in response to computer-readable instructions. The processing element 1808a may be a central processing unit of the analysis device 1800. Additionally or alternatively, the processing unit 1808a may be other processors within the device including application specific integrated chips (ASIC) and other microcontroller devices.

The memory 1812 may include a variety of types of non-transitory computer-readable storage media, including, for example, read access memory (RAM), read-only memory (ROM), erasable programmable memory (e.g., EPROM and EEPROM), or flash memory. The memory 1812 is configured to store computer-readable instructions, sensor values, and other persistent software elements. Computer-readable media 1816 may also include a variety of types of non-transitory computer-readable storage media including, for example, a hard-drive storage device, a solid state storage device, a portable magnetic storage device, or other similar device. The computer-readable media 1816 may also be configured to store computer-readable instructions, sensor values, and other persistent software elements.

In this example, the processing unit 1808a is operable to read computer-readable instructions stored on the memory 1812 and/or computer-readable media 1816. The computer-readable instructions may adapt the processing unit 1808a to perform the operations or functions described above with respect to FIGS. 2-16. The computer-readable instructions may be provided as a computer-program product, software application, or the like.

As shown in FIG. 18, the analysis device 1800 may also include a display 1818. The display 1818 may include a liquid-crystal display (LCD), organic light emitting diode (OLED) display, light emitting diode (LED) display, or the like. If the display 1818 is an LCD, the display may also include a backlight component that can be controlled to provide variable levels of display brightness. If the display 1818 is an OLED or LED type display, the brightness of the display 1818 may be controlled by modifying the electrical signals that are provided to display elements.

The analysis device 1800 may also include a battery 1824 that is configured to provide electrical power to the components of the analysis device 1800. The battery 1824 may include one or more power storage cells that are linked together to provide an internal supply of electrical power. In this regard, the battery 1824 may be a component of a power source 1828 (e.g., including a charging system or other circuitry that supplies electrical power to components of the analysis device 1800). The battery 1824 may be operatively coupled to power management circuitry that is configured to provide appropriate voltage and power levels for individual components or groups of components within the analysis device 1800. The battery 1824, via power management circuitry, may be configured to receive power from an external source, such as an AC power outlet or interconnected computing device. The battery 1824 may store received power so that the analysis device 1800 may operate without connection to an external power source for an extended period of time, which may range from several hours to several days.

The analysis device 1800 may also include one or more sensors 1840 that may be used to detect a touch and/or force input, environmental condition, orientation, position, or some other aspect of the analysis device 1800. In this regard, the sensors 1840 may be used to detect an input at a touch-sensitive display (e.g., display 1818) of the analysis device 1800 and/or other surface or feature, such as an external surface of the analysis device 1800 defined by an outer enclosure or shell. Example sensors 1840 that may be included in the analysis device 1800 may include, without limitation, one or more accelerometers, gyrometers, inclinometers, goniometers, or magnetometers. The sensors 1840 may also include one or more proximity sensors, such as a magnetic hall-effect sensor, inductive sensor, capacitive sensor, continuity sensor, or the like. Resistive and contact-based sensors may also be used.

The sensors 1840 may also be broadly defined to include wireless positioning devices including, without limitation, global positioning system (GPS) circuitry, Wi-Fi circuitry, cellular communication circuitry, and the like. As such, the sensors 1840 may be used to identify an environment of the analysis device 1800 (e.g., a clinical setting, a service facility, and so on). The analysis device 1800 may, in some embodiments, execute a different mode or configuration based on the identified environment, such as executing different analysis cycles, testing or calibrating produces, and so on. The analysis device 1800 may also include one or more optical sensors including, without limitation, photodetectors, photosensors, image sensors, infrared sensors, or the like. In one example, the sensor 1840 may be an image sensor that detects a degree to which an ambient image matches a stored image. As such, the sensors 1840 may be used to identify a user of the analysis device 1800. In this regard, the sensors 1840 may be used to control access to the analysis device 1800, for example, such as by initiating one or more operations when the sensors 1840 identify a known or authenticated user. The sensors 1840 may also include one or more acoustic elements, such as a microphone used alone or in combination with a speaker element. This may allow the analysis device 1800 to be operable by voice control, among other possibilities. The sensors 1840 may also include a temperature sensor, barometer, pressure sensor, altimeter, moisture sensor or other similar environmental sensor. The sensors 1840 may also include a light sensor that detects an ambient light condition of the analysis device 1800.

The sensors 1840, either alone or in combination, may generally be a motion sensor that is configured to determine an orientation, position, and/or movement of the analysis device 1800. For example, the sensors 1840 may include one or more motion sensors including, for example, one or more accelerometers, gyrometers, magnetometers, optical sensors, or the like to detect motion. The sensors 1840 may also be configured to determine one or more environmental conditions, such as temperature, air pressure, humidity, and so on. The sensors 1840, either alone or in combination with other input, may be configured to estimate a property of a supporting surface including, without limitation, a material property, surface property, friction property, or the like.

The analysis device 1800 may also include a camera 1832 that is configured to capture a digital image or other optical data. The camera 1832 may include a charge-coupled device, complementary metal oxide (CMOS) device, or other device configured to convert light into electrical signals. The camera 1832 may also include one or more light sources, such as a strobe, flash, or other light-emitting device. As discussed above, the camera 1832 may be generally categorized as a sensor for detecting optical conditions and/or objects in the proximity of the analysis device 1800. However, the camera 1832 may also be used to create photorealistic images that may be stored in an electronic format, such as JPG, GIF, TIFF, PNG, raw image file, or other similar file types. In a sample embodiment, the camera 1832 may be used to capture an image of an authenticated user of the analysis device 1800. The photorealistic image captured by the camera 1832 may be stored (e.g., at memory 1812 and/or an external source). The sensors 1840, as described above, may be used to compare an ambient image (e.g., a user requesting access) with the stored imaged. Where the images sufficiently match, the analysis device 1800 may allow the requesting user to initiate one or more operations (e.g., testing a breath sample). This may be helpful in clinical settings, for example, in which may be desirable to limit physical contact with the analysis device 1800.

The analysis device 1800 may also include a communication port 1844 that is configured to transmit and/or receive signals or electrical communication from an external or separate device. The communication port 1844 may be configured to couple to an external device via a cable, adaptor, or other type of electrical connector. In some embodiments, the communication port 1844 may be used to couple the analysis device 1800 with a computing device and/or other appropriate accessories configured to send and/or receive electrical signals. The communication port 1844 may be configured to receive identifying information from an external accessory, which may be used to determine a mounting or support configuration. For example, the communication port 1844 may be used to determine that the analysis device 1800 is coupled to a mounting accessory, such as a particular type of stand or support structure.

Other examples and implementations are within the scope and spirit of the disclosure and appended claims. For example, features implementing functions may also be physically located at various positions, including being distributed such that portions of functions are implemented at different physical locations. Also, as used herein, including in the claims, “or” as used in a list of items prefaced by “at least one of” indicates a disjunctive list such that, for example, a list of “at least one of A, B, or C” means A or B or C or AB or AC or BC or ABC (i.e., A and B and C). Further, the term “exemplary” does not mean that the described example is preferred or better than other examples.

The foregoing description, for purposes of explanation, uses specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A breath analysis system, comprising:
a breath capture component having an internal volume;
a cartridge attachable to the breath capture component and having a permeable membrane;
an analysis device having a column, a first reagent and a second reagent, the analysis device coupled with the cartridge; and
wherein the analysis device is configured to:
draw a breath sample held within the internal volume of the breath capture device through the permeable membrane;
elute the breath sample from the permeable membrane using the first reagent;
input the eluted breath sample to the column using the second reagent; and
detect one or more target compounds in the breath sample at an output of the column.

2. The breath analysis system of claim 1, wherein:
the breath capture component is an inflatable bag configured to receive the breath sample from a user.

3. The breath analysis system of claim 1, wherein:
the permeable membrane is composed of a bed of silica.

4. The breath analysis system of claim 1, wherein:
the column is composed of a high density silica configured to separate the target compounds in the breath sample based on the target compound's molecular weight.

5. The breath analysis system of claim 4, wherein:
the target compounds in the breath sample are target compounds having one or more carbonyl containing moieties.

6. The breath analysis system of claim 5, wherein:
the target compounds having one or more carbonyl containing moieties are aldehydes, ketones, carboxylic acids, or mixtures thereof.

7. The breath analysis system of claim 6, wherein:
the aldehydes are aliphatic aldehydes, di-aldehydes, aromatic aldehydes, or mixtures thereof.

8. The breath analysis system of claim 4, wherein:
the analysis device further comprises an optical detector;
the second reagent is a dye attachable to the one or more target compounds; and
the optical detector detects a value of the separated target compounds by measuring a fluorescence of the dye when hit by an excitation source.

9. The breath analysis system of claim 8, further comprising:
a display configured to depict a graphical output corresponding to the value.

10. A method for determining a target compound content in a breath sample, comprising:
drawing a breath sample through a permeable membrane connected to an analysis device;
eluting the breath sample from the permeable membrane using a first reagent from a container positioned within the analysis device;
advancing the eluted breath sample through a column using a second reagent from the container; and
detecting the target compound in the breath sample at an output of the column corresponding to the target compound content of the breath sample;
wherein the container comprises a quantity of the first reagent and the second reagent sufficient to determine the target compound content in the breath sample.

11. The method of claim 10, further comprising:
after the eluting and before the advancing, mixing the eluted breath sample with a fluorescent dye, wherein the fluorescent dye is configured to attach to the target compound in the breath sample to form fluoresced particles.

12. The method of claim 11, wherein the detecting further comprises:
propagating a laser through the output of the column; and
detecting the fluoresced particles by measuring an increase in brightness of the fluorescent dye.

13. The method of claim 12, further comprising:
blocking spectrum wavelength associated with the laser from reaching a detector used to measure the increase in brightness of the dye.

14. The method of claim 11, further comprising:
after the optical detector detects the value of the separated target compounds by measuring the fluorescence of the dye when hit by the excitation source;
drawing a second breath sample through the permeable membrane connected to the analysis device;
eluting the second breath sample from the permeable membrane using the first reagent from the container positioned within the analysis device:
advancing the eluted second breath sample through the column using the second reagent from the container; and
detecting the target compounds in the second breath sample at the output of the column corresponding to the target compound content of the second breath sample;
wherein the container comprises a quantity of the first reagent and the second reagent sufficient to determine the target compound content in the second breath sample.

15. An analysis device, comprising:
a sample capture module configured to retain target compounds from a breath sample;
a mixing module coupled to the sample capture module and configured to mix the retained target compounds with a group of first reagents;
an injection module separated from the mixing module and configured to form a pressurized combination of one or more second reagents and a buffer; and
a detection module configured to determine a value of the retained target compounds by:
receiving an output of the mixing module in a first configuration that loads a sample loop;
in response to loading a volume of the sample loop, receiving an output of the injection module in a second configuration that advances the loaded volume through a column; and
detecting a value for the retained target compounds.

16. The analysis device of claim 15, wherein:
the sample capture module is configured to form an elution having the retained target compounds; and
in response to a detection of the elution, the mixing module is configured to initiate the mixing of the second reagents and buffer with the elution having the retained target compounds.

17. The analysis device of claim 15, wherein, the injection module is configured to form the pressurized combination by combining the second reagent having a first flow rate with the buffer having a second flow rate into a common flow path.

18. The analysis device of claim 16, wherein:
the injection module is configured to detect a depressurization of the pressurized combination of the second reagent and the buffer; and
in response to the detection of the depressurization, the detection module is configured to initiate a third configuration that advances an output of the injection module to a waste outlet.

19. The analysis device of claim 16, wherein the one or more second reagents includes a fluorescent dye configured to react with the target compounds in the elution to form dye particles.

20. The analysis device of claim 19, wherein the detection module further comprises a laser and an optical detector, such that:
the laser exposes an output of the column to radiation that causes the dye particles to fluoresce and the detecting to be with the optical detector.
